(19) United States
(12) Patent Application Publication
(10) Pub. No.: US 2012/0245104 A1
(43) Pub. Date: Sep. 27, 2012

(54) NOVEL RETRO-INVERSO LEPTIN PEPTIDE ANTAGONISTS

(76) Inventor: Paul C. Leavis, Epping, NH (US)

(21) Appl. No.: 13/053,636

(22) Filed: Mar. 22, 2011

(51) Int. Cl. A61K 38/17; A61P 43/00

(52) U.S. Cl. 514/21.4

(57) ABSTRACT

Disclosed herein are peptides comprising a leptin sequence and methods for their use in preventing ObR signaling in a leptin-responsive cell. A leptin peptide of the present invention binds to but does not activate ObR signaling in a leptin-responsive cell, thereby inhibiting the up-regulatory effects of leptin on ObR signaling in the leptin-responsive cell. Administration of the peptide effectively prevents embryo implantation in a mammal to which the peptide has been administered. Also disclosed herein is a method for identifying a peptide antagonist of ObR, wherein the peptide comprises a leptin sequence. Further disclosed are truncations, modifications and retro-inversions of the peptides of the present invention.

### BACKGROUND OF THE INVENTION

Leptin (OB, the product of ob gene), is a pleiotropic molecule mainly secreted by white adipocytes that plays a relevant role in the regulation of body weight and food intake (for Review, see Gonzalez et al., Leptin and Reproduction, Hum Reprod Update, 6(3):290-300, 2000). In contrast to leptin, the leptin receptor (OB-R, the product of the db gene) has several spliced variants. The full-length and functional OB-R (OB-Rb) is expressed by the hypothalamus and plays a key role in the energy balance process. OB-R isoforms with shorter cytoplasmatic tail are expressed in many tissues, but their function remain unknown. A soluble OB-R that could regulate leptin biological actions has been also described. Leptin sequence is highly conserved in many species but some differences in OB-R sequences are found between species.

Binding of leptin to OB-R induces the homodimerization of the receptor that in turns allow the binding of Janus kinase 2 (JAK2) to specific box motifs in the intracytoplasmatic tail of OB-R. JAK2 phosphorylates OB-R followed by phosphorylation of signal transducer and activator of transcription 3 (STAT3), which, in turn dimerizes and translocates to the nucleus, thus activating several downstream signaling pathways. In addition to the JAK-STAT signaling pathway, other pathways, including the mitogen-activated protein kinase (MAPK), protein kinase C (PKC), and PI3-kinase pathways, are also activated by leptin.

Since the discovery of leptin in 1994 (Zhang et al., Nature. 1994 Dec. 1; 372(6505):425-432) emerging evidence have been accumulating that strongly link leptin signaling with the reproductive function (Gonzalez et al., Leptin and Reproduction, Hum Reprod Update. 2000 May-June; 6(3):290-300).

In vitro studies have shown that leptin and OB-Rb are expressed by female reproductive tissues, including ovaries, oocytes, preimplantation embryo, endometrium and placenta. Leptin can promote the development of mouse preimplantation embryos through OB-R signaling. Leptin protein has been found in human and mouse oocytes, and preimplantation embryos. But, leptin mRNA has been only found at blastocyst stage. Leptin at physiological concentrations could positively affect phosphorylation of STAT3 (p-STAT3) in mouse oocytes. These data suggest that OB-R signaling in oocytes and early preimplantation embryos up to morula stage would require maternal supply of leptin. A particular cell-borne pattern for leptin and STAT3 has been found in outer blastomers of human and mouse blastocysts. These data would indicate that leptin and OB-R are required to establish the cross-talk between the implanting embryo and the receptive endometrium.

In vitro the secretion of leptin is regulated by human preimplantation embryos co-cultured with endometrial cells (Gonzalez et al., Leptin and Reproduction, Hum Reprod Update, 6(3):290-300, 2000). Leptin induces the acquisition of the invasive phenotype of human trophoblast cells. Leptin increase the levels of β3-integrin (a marker of endometrial receptivity) in human endometrial epithelial cells (Gonzalez and Leavis, Endocrine, 16(1):21-28, 2001). Moreover, leptin in a dose-dependent manner increased p-STAT3 and leukemia inhibitory factor (LIF), interleukin-1 (IL-1) and levels of their cognate receptors in rabbit (Gonzalez and Leavis, Endocrine, 2003, 21(2):185-195, 2003) and human endometrial cells (Gonzalez et al., Endocrinology, 145(8):3850-3857, 2004; Epub 2004 May 13, 2004). Lastly, blockade of the OB-R with antibodies abrogated leptin-induced effects suggesting that leptin signaled through OB-R and the JAK/STAT3 pathways (Gonzalez et al., Mol Hum Reprod, 9(3):151-158, 2003; Gonzalez et al., Endocrinology, 145(8):3850-3857, 2004; Epub 2004 May 13, 2004).

Although the specific mechanisms whereby leptin modulates reproductive function are not completely understood leptin appears to be essential for normal preimplantation and/or implantation processes. Overall in vitro and in vivo data suggest that leptin signaling impact implantation capabilities in both entities: preimplantation embryo and endometrium.

In vivo studies have shown that mouse mutant deficient in leptin (ob/ob) (Zhang et al., 1994) or OB-R (db/db) are obese and infertile. Fertility can be restored in ob/ob by exogenous leptin. The withdrawal of leptin infusion in ob/ob females short after fertilization impairs implantation. Leptin injection into starved mice restores fertility. A postovulatory increase in serum leptin concentration appears to be associated with implantation potential and low expression of OB-R has been found in endometrium from women with unexplained infertility.

### SUMMARY OF THE INVENTION

The present invention provides peptide compositions, each composition comprising a leptin sequence and characterized by the ability to bind to but not activate the leptin receptor. The specific peptides disclosed are non-obvious modifications of sequences termed LPA-1 (IQKVQDDTKTLIKTIVTRINDISHTQSVSSKQ; SEQ ID NO:1) and LPA-2 (SRNVIQISNDLENLRDLLHVLAFSKS; SEQ ID NO: 2) that comprise sequences derived from helix I and helix III, respectively, of the human leptin sequence. Peptides of the present invention may be used to alter leptin-dependent functions in leptin-responsive cells.

The present invention provides a method for preventing ObR signaling in a leptin-responsive cell. The method comprises contacting a leptin-responsive cell with a peptide comprising a leptin sequence, modified leptin sequence or retro-inversion leptin sequence (herein after, “LPA sequence”). A peptide of the present invention comprising a leptin sequence is characterized by its ability to inhibit (partially or totally) the up-regulatory effects of leptin on ObR signaling in the leptin-responsive cell. In the present invention, inhibition in the leptin-responsive cell is relative to an identical leptin-responsive cell which has not been contacted with the peptide.

In the present invention, contact of the leptin-responsive cell with a peptide comprising a leptin sequence may be in vitro or in vivo. The cell may be a human or rabbit cell or other mammalian cell, and may further be an endometrial or other leptin-responsive cell. In the context of the present invention, the endometrial cell may be of epithelial or stromal cell types. A peptide of the present invention is to be delivered in an amount effective to bind but not activate ObR in cells where inhibition of ObR signaling is desirable.

A peptide of the present invention may be administered to a mammal to alter any leptin-dependent function in the mammal. In a preferred embodiment, administration of the peptide results in the prevention of embryo implantation in the mammal. Administration of a peptide comprising a leptin sequence in the present invention may be to any mammal including a human, mouse or rabbit.

A method for preventing ObR signaling in a leptin-responsive cell may comprise inhibiting the up-regulatory effects of leptin on a signaling event downstream of ObR. The signaling event downstream of the leptin receptor may be p-Stat-3, β3-integrin, IL-1 and/or LIF signaling. The inhibition of ObR signaling may result in an inhibition of the up-regulatory effects of leptin on the expression of a leptin-sensitive target. The leptin-sensitive target may be p-Stat-3, β3-integrin, IL-1R tl, LIF-R (leukemia inhibitory factor receptor), LIF, IL-1β and/or IL-1Ra.

Also provided herein is a method for identifying a peptide antagonist of ObR wherein the peptide antagonist of ObR comprises a leptin sequence. The method comprises first providing a leptin or leptin equivalent sequence. The method further comprises superimposing the three-dimensional structure of the provided leptin or leptin equivalent sequence on the three-dimensional structure of the G-CSF/G-CSF R complex. The method further comprises identifying a region of the provided leptin or leptin equivalent sequence which superimposes on the residues of G-CSF that interact with G-CSF R in a G-CSF/G-CSF R three-dimensional structure. The method further comprises synthesizing a peptide comprising a leptin or leptin equivalent sequence which corresponds to the identified leptin or leptin equivalent region and testing the peptide in cell culture for its ability to inhibit (partially or totally) the up-regulatory effects of leptin on ObR signaling in a leptin-responsive cell. In a preferred embodiment, a leptin region identified in this method may comprise helix I or helix III of the leptin sequence.

### BRIEF DESCRIPTION OF THE DRAWINGS

FIG. 1 shows sequences of LPA-1 leptin derived full-length and truncated peptides.

FIG. 2 shows sequences of LPA-2 leptin derived full-length and truncated peptides.

FIG. 3 shows inhibition of MCF-7 leptin-induced proliferation by truncated LPA-1 peptides.

FIG. 4 shows inhibition of MCF-7 leptin-induced proliferation by truncated LPA-2 peptides.

FIG. 5 shows a helical wheel representation of LPA-1 [6-24], sequences of modified peptides and sequence of scrambled control peptide.

FIG. 6 shows a helical wheel representation of LPA-2 [72-89], sequences of modified peptides and sequence of scrambled control peptide.

FIG. 7 shows IC50 data of modified peptides in MCF-7 and HT29 leptin-induced activity as well as IC50 data of retro-inverso LPA-1 [24-6; SEQ ID NO: 31] and LPA-2 [89-72; SEQ ID NO: 32] peptides.

### DETAILED DESCRIPTION OF THE PRESENT INVENTION

The present invention is based on the finding that peptides comprising a leptin sequence are capable of regulating leptin receptor (Ob-R) function. More specifically, the invention is based on the observation that peptides comprising a leptin sequence bind to the leptin receptor and blocks its activation. Such peptides may be utilized in controlling leptin-dependent functions, including but not limited to embryo implantation. The leptin sequence can be further exploited in identifying other peptides which act as potent inhibitors of leptin receptor function.

Further, the present invention is related to modified versions of the active peptides of the present invention. The invention contemplates non-identical equivalent compositions of the present invention that may include the introduction of conservative amino acid substitutions, deletions, and/or additions into the peptide comprising a leptin sequence, as long as the substitutions, deletions, and/or additions do not substantially prevent the binding of the peptide to ObR and do not negatively affect the antagonistic properties of the peptide. Non-conservative amino acid substitutions are also contemplated. Conservative substitutions are generally defined in the art and defined herein as amino acid replacements that are similar with respect to functional and structural characteristics of the original amino acid. For example, the replacement of one polar amino acid with another polar amino acid would be a conservative substitution. Non-conservative substitutions are generally defined in the art and defined herein as amino acid replacements that are not similar with respect to the structure or function of the original amino acid. Further still, the invention relates to retro-inverso variants of the active peptides of the present invention.

The stereochemistry of polypeptides can be described in terms of the topochemical arrangement of the side chains of the amino acid residues about the polypeptide backbone which is defined by the peptide bonds between the amino acid residues and the .alpha.-carbon atoms of the bonded residues. In addition, polypeptide backbones have distinct termini and thus direction.

The vast majority of naturally occurring amino acids are L-amino acids. Naturally occurring polypeptides are largely comprised of L-amino acids. D-amino acids are the enantiomers of L-amino acids and form peptides which are herein referred to as inverso peptides, that is, peptides corresponding to native peptides but made up of D-amino acids rather than L-amino acids.

Retro peptides are made up of L-amino acids in which the amino acid residues are assembled in opposite direction to the native peptide sequence. Retro-inverso modification of naturally occurring polypeptides involves the synthetic assemblage of amino acids with α-carbon stereochemistry opposite to that of the corresponding L-amino acids, i.e., D- or D-allo-amino acids, in reverse order with respect to the native peptide sequence. A retro-inverso analogue thus has reversed termini and reversed direction of peptide bonds while approximately maintaining the topology of the side chains as in the native peptide sequence.

Processes for synthesis of retro-inverso peptide analogues including some processes for the solid-phase synthesis of partial retro-inverso peptide analogues have been described (Bonelli, et al., Int J Pept Protein Res. 1984 December; 24(6):553-556).

Importantly, it has been observed that due to the stereospecificity of enzymes with respect to their substrates as well as specificity of receptor/ligand interactions, replacement of L-amino acid residues with D-amino acid residues in peptide substrates generally abolishes proteolytic enzyme recognition and/or binding activity. Reversal of the amino acid sequence can exasperate this problem. Therefore, those of ordinary skill in the art have had little success in creating retro-inverso peptides with significant activity. Retro-inverso peptides with activity equivalent to native sequences are virtually unheard of in the art. Herein are described retro-inverso leptin peptide antagonists showing equivalent, near-equivalent or increased activity as compared to unmodified sequences.

Advantages of the retro-inverso peptides of the present invention include greater stability by being refractory to proteolysis by endogenous enzymes and, therefore, having greater bioavailability as a therapeutic agent. Greater bioavailability allows for the use of lower and less frequent doses. Further, greater stability results in longer shelf life.

The present invention relates to methods and compositions for inhibiting Ob-R signaling in a leptin-responsive cell. More specifically, the present invention relates to methods and compositions for inhibiting Ob-R signaling in a leptin-responsive cell comprising contacting the leptin-responsive cell with a peptide comprising a retro-inverso leptin sequence or a truncated and-or modified leptin sequences based on helix I or helix III of the leptin sequence. In embodiments of the present invention, the peptide comprising a retro-inverso leptin sequence of the present invention acts as an antagonist of Ob-R in the leptin-responsive cell as well as or better than the un-engineered versions. The peptide competes with the native leptin ligand for binding to its receptor, or alternatively displaces the native leptin ligand from an already bound receptor. As exemplified, a peptide of the present invention binds Ob-R with high affinity and specificity. A peptide for use in the methods of the present invention inhibits (partially or totally) the up-regulatory effects of leptin on Ob-R signaling in the leptin-responsive cell, the leptin-responsive cell being relative to an identical leptin-responsive cell which has not been contacted with the peptide.

Methods disclosed herein for inhibiting Ob-R signaling in a leptin-responsive cell may be carried out in vitro. Inhibition of Ob-R signaling can be effectively achieved in a leptin-responsive cell in tissue culture. Ob-R signaling is conserved in a majority of mammalian species, and, as such, methods for inhibiting the same may be carried out in tissue culture cells derived from a majority of mammals. Examples of mammalian cell lineages in which Ob-R signaling is conserved include those derived from a human, mouse, or rabbit. Additionally, Ob-R signaling is conserved in cells derived from an array of tissue types, and as such, methods of the present invention may be carried out in cells derived from the same. Non-limiting examples of cell types include endometrial, follicular, adipose, placental, oocyte, preimplantation embryo, and fetal cell types. Specific endometrial cell types in which Ob-R signaling is conserved include endometrial epithelial and endometrial stromal cells. The methods for inhibiting Ob-R signaling, as disclosed herein, may be practiced using any cell which expresses the leptin receptor. The leptin receptor may be endogenous or exogenous to the cell. In vitro, the methods disclosed herein for inhibiting Ob-R signaling may be used to study the actions of leptin in a variety of cell types, or for the development of therapeutics for modifying Ob-R signaling. More specifically, the methods of the present invention may be used to study the roles of Ob-R signaling in reproduction, inflammation, proliferation, apoptosis, and angiogenesis. The methods of the present invention may be used to study and/or develop therapeutics for disease states relating to leptin misregulation, including but not limited to diabetes mellitus, acquired immune disease, cancer (including endometrial cancer), endocrine disorders of the adrenal cortex and pituitary glands, preeclampsia, endometriosis, polycystic ovarian syndrome, fertility, and obesity. The methods disclosed herein are not intended to be limited only for use with cells in culture.

Methods disclosed herein for inhibiting Ob-R signaling in a leptin-responsive cell may also be carried out in vivo. Inhibition of Ob-R signaling can be effectively achieved in cells within an organism. Inhibition of Ob-R signaling in cells within an organism may be achieved wherein the mammal is human or non-human. Ob-R signaling is conserved in a majority of mammals, and, as such, methods for inhibiting the same may be carried out in a variety of mammals. Examples of mammals in which Ob-R signaling is conserved include humans, mice, and rabbits. The methods disclosed herein are not intended to be limited only for use with cells in any particular tissue or mammal. In vivo, the methods disclosed herein for inhibiting Ob-R signaling may be used to alter leptin-dependent functions and/or leptin/Ob-R misregulation.

In one aspect, the present invention relates to methods for inhibiting Ob-R signaling in a leptin-responsive cell, the method comprising administering to a mammal a therapeutically effective amount of a peptide of the present invention comprising a retro-inverso leptin sequence or a truncated and-or modified leptin sequences based on helix I or helix III of the leptin sequence. In methods of the present invention, the peptide binds to, but does not activate, Ob-R signaling in a leptin-responsive cell of the mammal to which the peptide has been administered. The peptide effectively binds to Ob-R without transmitting the cellular signals that characterize the binding of leptin to Ob-R, thereby inhibiting Ob-R signaling in the treated mammal, the inhibition being relative to a matched mammal to which the peptide has not been administered, and also to a matched animal to which a scrambled version of the peptide has been administered.

The mammal to which the peptide is administered in the present invention may be any mammal which has genes that encode leptin and leptin receptor molecules or equivalents. An equivalent may be any protein bearing structural homology to a known leptin molecule, which is postulated to bind a protein bearing structural homology to a known leptin receptor molecule. The specific peptides disclosed may be administered to any mammal with a conserved leptin sequence. Examples of mammals with known conserved leptin sequences include humans, mice, and rabbits.

It is a further object of the present invention to provide methods for controlling leptin-dependent functions. In a preferred embodiment, administration of a peptide comprising a retro-inverso leptin sequence prevents embryo implantation in a mammal to which the peptide has been administered. A peptide comprising a retro-inverso leptin sequence may further be used to inhibit leptin actions in treating conditions characterized by abnormalities of leptin metabolism. Non-limiting examples of such conditions include endometriosis, diabetes mellitus, acquired immune disease, cancer, endocrine disorders of the adrenal cortex and pituitary glands, preeclampsia, endometriosis, polycystic ovarian syndrome and reduced fertility.

It is an object of the present invention to administer a peptide comprising a retro-inverso leptin sequence or other leptin peptide of the present invention in a physiologically acceptable carrier in a therapeutically effective amount. A therapeutically effective amount is defined as any amount which is sufficient for inhibiting Ob-R signaling in a desired cell. A peptide of the present invention may be administered alone or in a combination with other pharmaceutical therapies. Pharmaceutical compositions of the present invention may comprise one or more of the peptides of the present invention. Administration of said peptide or peptides to a mammal may be either local or systemic and may be for short or long durations. A peptide or peptides of the present invention may be delivered to a mammal intramuscularly, or alternatively, by intravenous, subcutaneous, or oral administration. Within embodiments of the invention, the compositions described herein may be administered as part of a sustained release implant.

Because the therapeutic target of the present invention is extracellular, delivery of a peptide of the present invention need only be to the extracellular surface of a leptin-responsive cell. It is not a requirement of the present invention that a peptide comprising a retro-inverso leptin sequence be delivered intracellularly. In the methods of the present invention, the pharmaceutical challenge of targeted intracellular delivery, therefore, is overcome. In the present invention, a peptide comprising a leptin sequence of the present invention is delivered to the extracellular surface of a leptin-responsive cell in which inhibition of Ob-R signaling is desired.

Until recently, the only demonstrated method for inhibiting Ob-R signaling in a cell comprised contacting a leptin-responsive cell with an effective amount of a functional antibody to Ob-R, the antibody characterized by its ability to prevent leptin binding to Ob-R. Blockade of Ob-R with a polyclonal antibody against the extracellular domain is known to inhibit the rate of formation of expanded mouse blastocyst and hatched blastocyst in an in vitro model of embryo development. In the Exemplification section that follows, blockade of Ob-R with an anti-Ob-R antibody was determined to inhibit the up-regulatory effects of leptin in endometrial cell culture. However, relatively high doses of antibodies were required to block Ob-R, which could potentially generate undesired immunological side effects in vivo. In contrast, in the methods of the present invention relatively low doses (3-300 nM) of a peptide comprising a retro-inverso leptin sequence or a truncated and-or modified leptin sequences based on helix I or helix III of the leptin sequence were determined as sufficient for blocking Ob-R function in vitro and, therefore, for preventing mouse embryo implantation in vivo. The methods of the present invention therefore have a distinct therapeutic advantage over antibody-based methods.

It is a further object of the present invention to provide methods for inhibiting Ob-R signaling in a leptin-responsive cell wherein the inhibition of Ob-R signaling in the cell results in an inhibition of the up-regulatory effects of leptin on a signaling event downstream of Ob-R. Leptin binding to its receptor promotes JAK-2 activation and triggers the phosphorylation of signal transducer and activator of transcription 3 (Stat3) that, in turn, activates a number of downstream signaling pathways. Blockade of Ob-R by a peptide comprising a leptin sequence of the present invention will inhibit activation of a number of downstream events in both human and rabbit cells and other mammalian cells. More specifically, a peptide comprising a leptin sequence of the present invention will inhibit p-Stat-3, β3-integrin, IL-1 and LIF signaling. It is an object of the present invention that a peptide be used to inhibit any of the signaling events that arise from leptin binding to Ob-R. The signaling event may include regulation of gene expression, phosphorylation, and/or secretion of a direct or indirect target of Ob-R. It is a requirement that the signaling event inhibited be leptin-sensitive and downstream of Ob-R. Non-limiting examples of leptin-sensitive targets of ObR inhibited by the methods of the present invention include p-Stat-3, β3-integrin, IL-1R tl, LIF-R, LIF, IL-1β and IL-1Ra.

It is a further object of the present invention to provide a method for identifying a peptide antagonist of Ob-R. The method comprises first providing a leptin or leptin equivalent sequence from a mammalian species. A leptin equivalent sequence may be any sequence which bears structural homology to a known leptin molecule, which is postulated to bind a protein bearing structural homology to a known leptin receptor molecule. A structural model of the provided leptin or leptin equivalent sequence bound to its receptor is then built. This may be accomplished by superimposing the three-dimensional structure of the leptin sequence on the three-dimensional structure of the G-CSF/G-CSF R complex. The structure of the leptin receptor complex is determined using any of a number of molecular modeling software programs available in the art. By comparing the residues of the G-CSF ligand that interact with G-CSF R, a determination of the region(s) of the leptin or leptin equivalent sequence which interact with its receptor can be made. A ligand/receptor complex other than G-CSF/G-CSF R may be utilized in identifying a peptide antagonist of Ob-R, as long as the complex bears presumptive structural homology to a leptin/Ob-R complex. Alternatively, identification of a peptide antagonist of Ob-R may be determined by a direct analysis of interacting residues of a leptin bound to Ob-R if the three-dimensional structure of such a complex is known. A peptide bearing the identified region of the leptin or leptin equivalent sequence is then synthesized and tested for its ability to inhibit ObR signaling in a cell. In a preferred embodiment, the peptide is tested in cell culture for its ability to bind to Ob-R and inhibit the up-regulatory effects of leptin on Ob-R signaling in a leptin responsive cell. It is a requirement that the identified peptide possesses a high binding affinity for Ob-R and be an effective antagonist of Ob-R. In a preferred embodiment, an identified leptin region comprises, consists of or consists essentially of any of residues 3-34 (LPA-1; SEQ ID NO: 1) of the leptin sequence (helix I) or any of residues 70-95 (LPA-2; SEQ ID NO: 2) of the leptin sequence (helix III) or a retro and/or inverso leptin sequence or a truncated and-or modified leptin sequences based on helix I or helix III of the leptin sequence. This method may include introducing conservative amino acid substitutions, deletions and/or additions into the identified region, as long as the substitutions, deletions and/or additions do not substantially prevent the binding of the peptide to ObR and do not negatively affect the antagonistic properties of the peptide. Further, this method may include introducing non-conservative amino acid substitutions that do not substantially prevent the binding of the peptide to ObR and do not negatively affect the antagonistic properties of the peptide. There are no strict requirements regarding the length of a peptide identified by this method.

Peptide compositions for use in the methods of the present invention are also herein provided. A composition of the present invention is characterized by its ability to inhibit the up-regulatory effects of leptin on ObR signaling in a leptin responsive cell. In a preferred embodiment, the composition comprises, consists of or consists essentially of a peptide comprising a retro-inverso leptin sequence. The leptin sequence confers upon the peptide the ability to bind Ob-R with a relatively high affinity and specificity and be an effective antagonist of ObR. In one embodiment, the peptide comprises, consists of or consists essentially of residues 3-34 (LPA-1; SEQ ID NO: 1) of the leptin sequence (helix I) or a retro and/or inverso leptin sequence or a truncated and/or modified leptin sequences based on helix I of the leptin sequence. In another embodiment, the peptide comprises residues, consists of or consists essentially of 70-95 (LPA-2; SEQ ID NO: 2) of the leptin sequence (helix III) or a retro and/or inverso leptin sequence or a truncated and-or modified leptin sequences based on helix III of the leptin sequence. A non-identical equivalent is a peptide comprising a sequence substantially similar to either SEQ ID NO:1 or SEQ ID NO:2, wherein the non-identical equivalent retains the functional properties of SEQ ID NO:1 or SEQ ID NO:2. Functional properties of the peptides (sequences) of the present invention include an ability to 1) compete with leptin for binding to Ob-R on a leptin-responsive cell and 2) block the leptin-dependent signaling in the leptin-responsive cell. A non-identical equivalent composition of the present invention may include the introduction of conservative amino acid substitutions, deletions and/or additions into the peptide comprising a leptin sequence, as long as the substitutions, deletions and/or additions do not substantially prevent the binding of the peptide to ObR and do not negatively affect the antagonistic properties of the peptide. Further, the compositions may include introducing non-conservative amino acid substitutions that do not substantially prevent the binding of the peptide to ObR and do not negatively affect the antagonistic properties of the peptide. Further still, compositions may include retro and/or inverso modifications based on helix I or helix III of the leptin sequence. There are no strict requirements regarding the length of a peptide composition comprising a leptin sequence for use in the methods of the present invention.

In the context of the present invention, the terms “inhibit,” “inhibition” and the like may refer to partial or total inhibition.

### EXEMPLIFICATION

### Example 1

### Creation of Smaller, More Effective Leptin Signaling Antagonists Based on LPA-1 and LPA-2

This invention encompasses retro-inverso compositions derived from peptides referenced as LPA 1 [SEQ ID NO: 1] and LPA-2 [SEQ ID NO: 2]. In this regard, this invention encompasses peptides that are shorter, more effective and of greater in vivo stability than the original LPA peptides described by Gonzalez and Leavis (Gonzalez, R. R. and Leavis, P. C., A peptide derived from the human leptin molecule is a potent inhibitor of the leptin receptor function in rabbit endometrial cells, Endocrine 21; 185-195 (2003) and U.S. Pat. Nos. 7,407,929 and 7,612,043; the contents of which are incorporated herein by reference in their entirety). In addition, this invention relates to LPA peptides that contain non-conservative amino acid substitutions and that are synthesized as retro-inverso peptides.

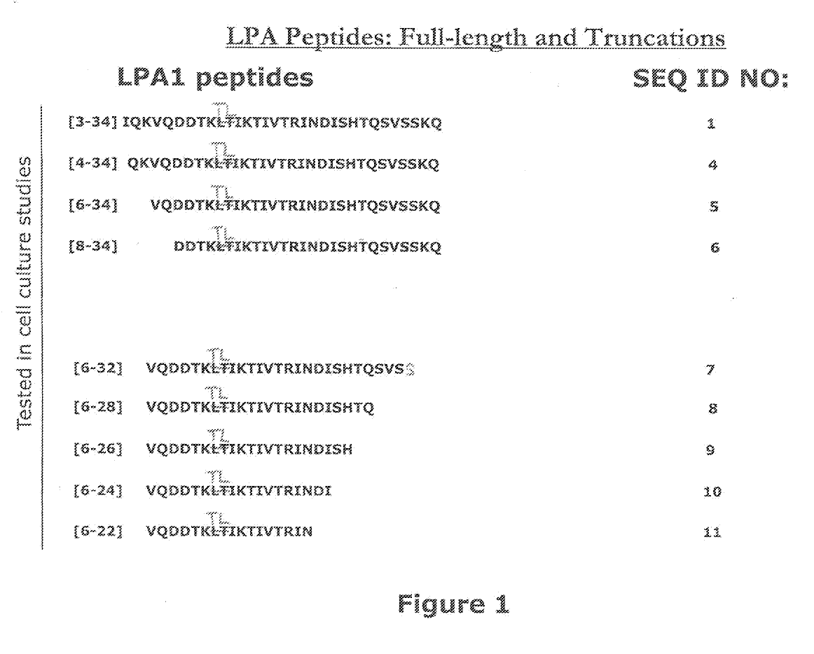

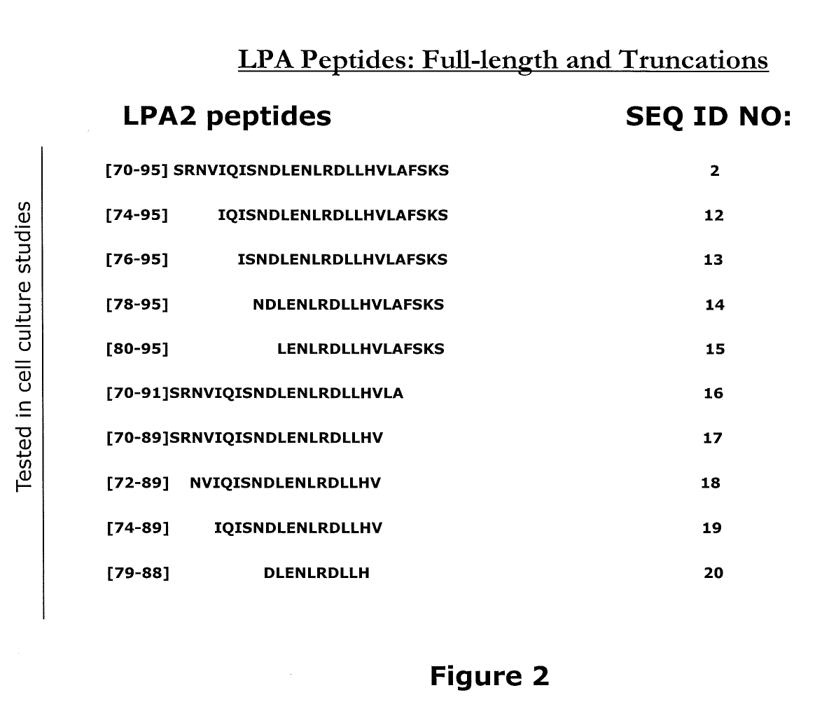

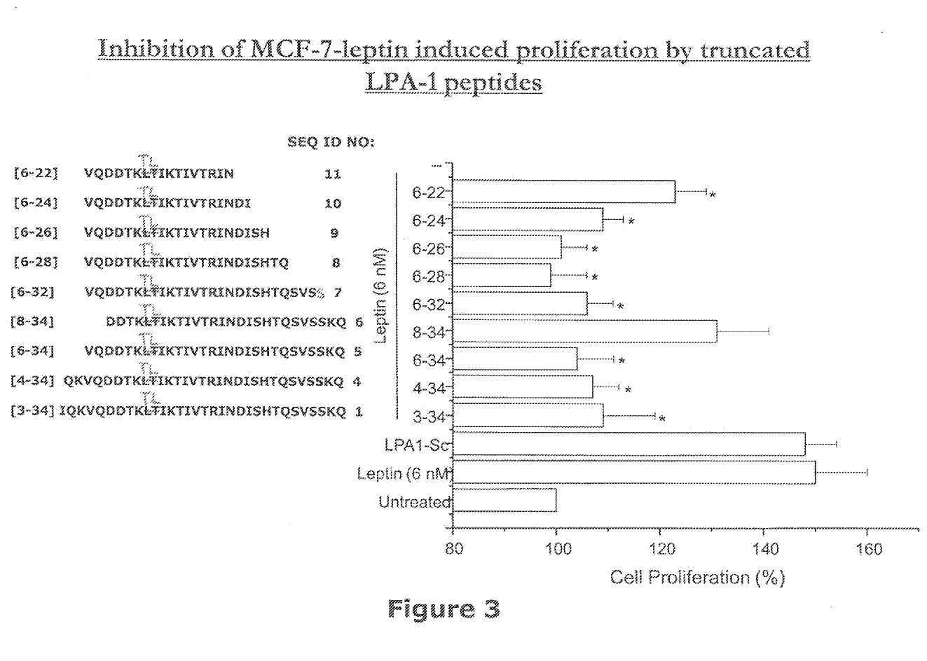

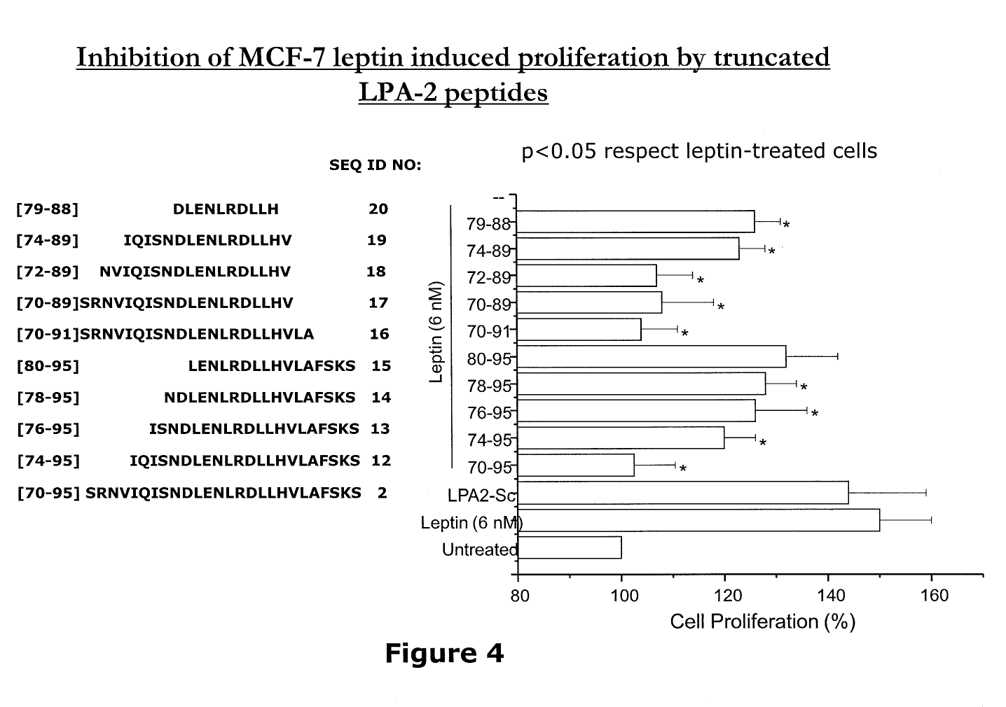

1) Truncation of LPA 1 [SEQ ID NO: 1] and LPA-2 [SEQ ID NO: 2]: Truncated versions of LPA-1 (full length corresponds to residues 3-34 of the leptin molecule) in which pairs of amino acids were removed sequentially from the N-terminus of the peptide resulting in peptides corresponding to leptin residues 4-34, 6-34 and 8-34. These peptides were compared to full length LPA-1 with respect to their abilities to inhibit leptin-induced cell growth. When the removal of an amino acid from the N-terminus resulted in a loss of inhibition we employed a similar approach to synthesize a series of C-terminally truncated peptides, all starting at the new active N-terminus and sequentially removing C-terminal amino acids until function is lost or decreased (leaving peptides corresponding to leptin residues 6-32, 6-28-6-26-6-24 and 6-22). Sequences of these peptides are shown in FIGS. 1. Activity results of these peptides with respect to full length LPA-1 and scrambled LPA-1 are shown in FIG. 3. The same approach was employed with LPA-2 creating truncated versions of LPA-2 (full length corresponds to residues 70-95 of the leptin molecule). The shortened LPA-2 peptides corresponded to leptin residues 74-95, 79-95, 78-95, 80-95, 70-91, 70-89, 72-89, 74-89 and 79-88). Sequences of these peptides are shown in FIG. 2. Activity results of these peptides with respect to full length LPA-2 and scrambled LPA-2 are shown in FIG. 4. Another LPA-2 scrambled peptide that may be used is VAEVLNRSDLIQRISFSLDLNNSKLH [SEQ ID NO: 3]. Based on the results given in FIGS. 3 (LPA-1) and 4 (LPA-2) (and other similar data not shown) LPA-1 truncation peptide corresponding to residues 6-24 of the leptin molecule and LPA-2 truncation peptide corresponding to residues 72-89 of the leptin molecule were chosen for further modification and study.

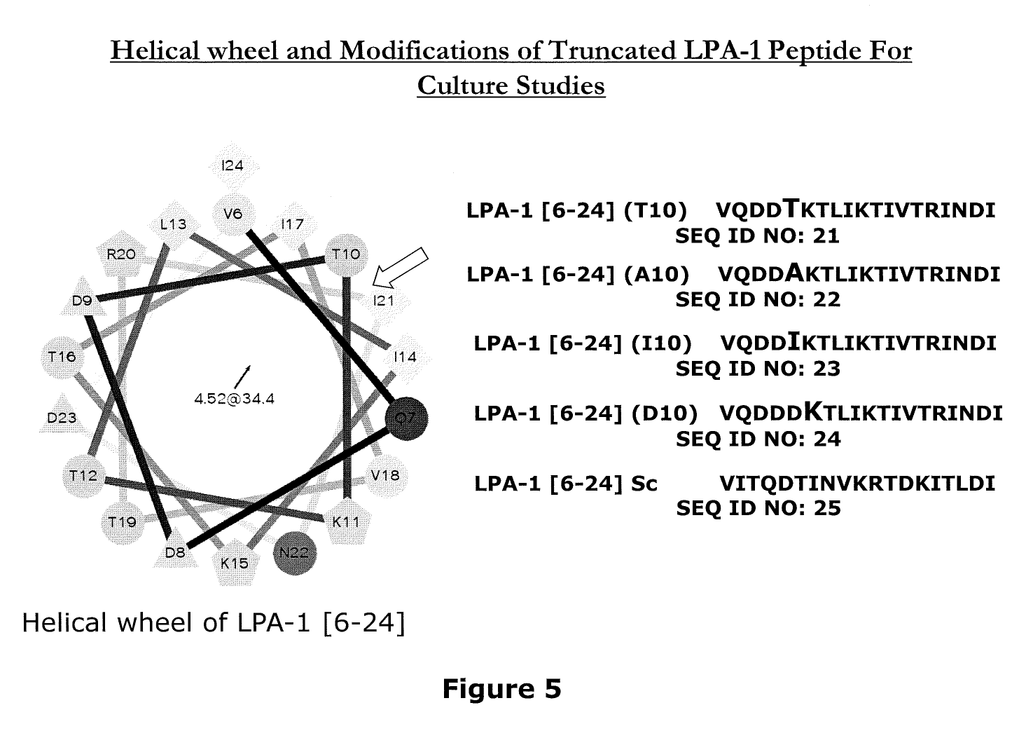

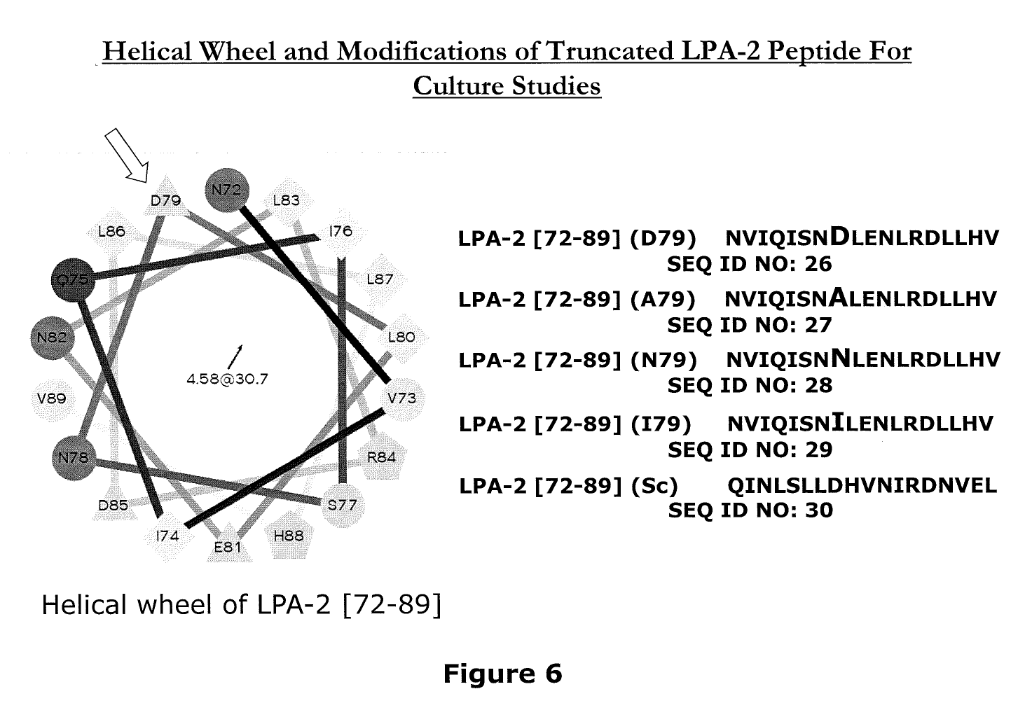

2) Non-conservative amino acid substitutions: A helical wheel projection of LPA-1 (4-24) is shown in FIG. 5. A helical wheel projection of LPA-2 (75-90) is shown in FIG. 6. A helical wheel is known in the art as a visual representation used to illustrate the properties of alpha helices in proteins. The sequence of amino acids that make up a helical region of the protein's secondary structure are plotted in a rotating manner where the angle of rotation between consecutive amino acids is 100°, so that the final representation looks down the helical axis. The plot reveals whether hydrophobic amino acids are concentrated on one side of the helix, usually with polar or hydrophilic amino acids on the other. This arrangement is common in alpha helices within globular proteins, where one face of the helix is oriented toward the hydrophobic core and one face is oriented toward the solvent-exposed surface. Specific patterns characteristic of protein folds and protein docking motifs are also revealed, as in the identification of leucine zipper dimerization regions and coiled coils.

Diamond-shaped residues indicate the hydrophobic surface of the helical peptide which is the putative binding site to the leptin receptor (ObR). All residues are highly hydrophobic except for the polar residue, T10 (threonine at residue 10) of LPA-1 and polar residue D79 (aspartic acid at residue 79) of LPA-2. In an effort to enhance binding/inhibitory activity of the peptide, we prepared substitutions of T10 and D79 with the hydrophobic residues, A (alanine), L (leucine), I (isoleucine) and V (valine). The results in Table 1, below, are IC50 (half maximal inhibitory concentration) values in nM for both LPA-1 and LPA-2.

TABLE 1
IC50 values in nM

| | | | |
|---|---|---|---|
| Full-length LPA-1 (3-34) | 39.1 | Full-length LPA-2 (70-95) | 33 |
| Truncated peptides (6-24) | | Truncated peptides (72-89) | |
| T10 (original amino acid) | 25.7 | D79 (original amino acid) | 32 |
| Ala substitution | 88 | Ala substitution | 54 |
| Ile substitution | 21 | Ile or Leu substitution | 16 |
| Val substitution | 20.7 | Val substitution | 15.6 |

In both cases (non-conservative amino acid substitutions in LPA-1 and LPA-2), replacement of the polar residue with hydrophobic residues Leu, Ile or Val reduce the IC50 values by a factor of 2. However, this result was not consistent across the entire group of hydrophobic amino acids tested as substitution with the hydrophobic amino acid alanine unexpectedly resulted in increased IC50 values thereby showing that the results were unpredictable to one of ordinary skill in the art.

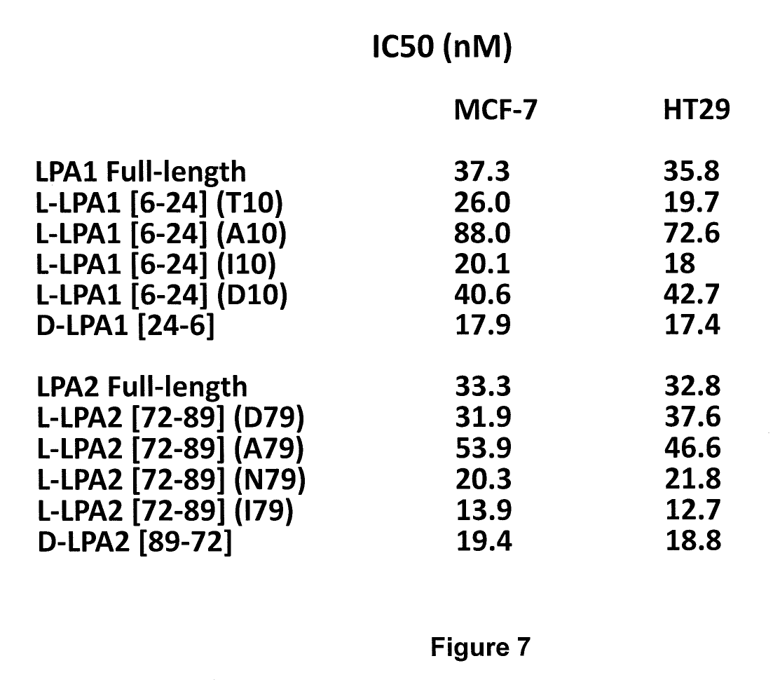

3) Retro-inverso peptides: The above peptides [LPA-1 (6-24) and LPA-2 (72-89)] containing the hydrophobic substitutions described above (with the exception of the alanine substitution) were synthesized as retro-inverso peptides as exemplified below. Retro-inverso peptides are peptides constructed with the reverse sequence and containing all D-amino acids. Our peptides exhibited the same side chain spatial arrangement as native peptides and, thus, contrary to conventional wisdom that retro-inverso peptides abolish recognition of substrate to ligand, we predicted that binding to ObR would be preserved (see IC50 values below). Further, we predicted that because our retro-inverso peptides consist of D-amino acids they would be refractory to proteolysis by endogenous enzymes and would more bioavailable as a therapeutic agent. FIG. 7 shows inhibition of leptin-induced growth at various peptide concentrations for the retro-inverso peptides without hydrophobic substitutions. Retro-inverso LPA-1(24-6) sequence IDNIRTVITKILTKTDDQV [SEQ ID NO: 31] and retro-inverso LPA-2(89-72) VHLLDRLNELDNSIQIVN [SEQ ID NO: 32]. For SEQ ID NOs: 31 and 32 both L (inverso) and D (retro-inverso) amino acid peptides are contemplated.

Unexpectedly, the D-peptides with hydrophobic substitutions provide inhibition equal to or greater than the original peptides. Studies were carried out using both MCF-7 and HT-29 cells. Results shown in Table 2 were for MCF-7 cells. The results with HT-29 cells were equivalent.

TABLE 2
IC50 values in nM.

| | | | |
|---|---|---|---|
| Full-length LPA-1 (3-34) | 39.1 | Full-length LPA-2 (70-95) | 33 |
| Truncated peptides (6-24) | | Truncated peptides (72-89) | |
| T10 (original amino acid) | 25.7 | D79 (original amino acid) | 32 |
| LPA1-D-peptides (6-24) | | LPA2-D-Peptides (72-89) | |
| D-T10(1) | 16.9 | D-D79(1) | 19.4 |
| D-I10 | 17.6 | D-I79 | 9.7 |
| D-L10 | 11.8 | D-L79 | 18.7 |
| D-V10 | 17.4 | D-V79 | 13.6 |

Conclusions: The peptides indicated all have the following improvements: 1) they are shorter and easier to make; 2) For the same molar equivalents, they require less material; 3) they all have substitutions and are therefore “engineered” peptides—not natural. Further, the substitutions are non-conservative substitutions and, therefore, provide unexpected results; 4) In the case of the D-amino acid peptides, they are more bioavailable and not degraded by serum enzymes. Further, success with D-amino acid peptides would have been unpredicted by one of ordinary skill in the art.

4) PEGylated peptides: PEGylation was carried out using mPEG-maleimide (20 kDa) which was attached to LPAs containing an N-terminal spacer peptide consisting of the residues GSWC [SEQ ID NO.: 33]. Studies using peptides PEGylated with this approach indicated that they exhibited the same activity as non-PEGylated peptides with the added benefit of increased water solubility.

Examples of peptides:

Inverso-LPA-1 NH₂-IDNIRTVITKILTKXDDQV-CONH₂ [SEQ ID NO.: 34], where X is selected from amino acids T, I, L and V. Both L (inverso) and D (retro-inverso) amino acid peptides are contemplated.

Inverso-LPA-2 NH₂-VHLLDRLNELXNSIQIVN-CONH₂ [SEQ ID NO.: 35], where X is selected from amino acids D, I, L and V. Both L (inversion) and D (retro-inverso) amino acid peptides are contemplated.

### Materials and Methods

Synthesis:

Peptides were synthesized by solid state peptide synthesis either manually or using an automated peptide synthesizer (ABI Model 433A). The peptide was synthesized on NovaPeg Rink Amide resin (Novabiochem). Fluorenylmethoxycarbonyl (Fmoc) was employed to block α-amino groups. Reactive side chains were blocked by a variety of available acid-labile protecting groups. Coupling of protected amino acids to the nascent peptide was accomplished by converting their α-carboxyl groups to active benzotriazole esters using the coupling reagent 2-(1H-benzotriazole-1-yl)-1,1,3,3-teteramethyluronium hexafluorophosphate (HBTU). Fmoc removal and coupling efficiency was monitored during synthesis by performing a ninhydrin (Kaiser) test on resin samples to detect the presence of free amines.

Cleavage and Post-Synthesis Workup:

The completed peptide was simultaneously cleaved from the resin and deprotected using a 95% trifluoroacetic acid (TFS) cocktail containing the scavenger molecules phenol, thioanisole and ethanedithiol. Cleavage was performed at room temperature for 3 hours with stirring. The cleaved peptide was precipitated in cold diethylether, sedimented by centrifugation and washed several times with ether to remove scavenger complexes with cleaved side-chain protecting groups. Final purification was accomplished by preparative reversed phase HPLC (Clipeus C18, 10 Ξm; 205×20 mm, Higgins Analytical) using a 0.2% trifluouroacetic acid acetonitrile/0.2% trifluouroacetic acid water solvent system. The quality and identity of the purified product was ascertained by:
i) MALDI-TOF mass spectrometry to verify the peptide mass
ii) Analytical rpHPLC (reverse-phase HPLC) to check purity

Scrambled Peptides:

For each LPA peptide we also designed and synthesized scrambled peptides: viz. peptides with the same amino acid composition but randomized sequence, as controls. The scrambled peptides were checked against data bases of know peptide/protein sequences to make sure that their sequences don't inadvertently correspond to any known protein nor possess a secondary structure similar to its active homolog.

Pegylation:

All peptides to be conjugated to PEG were synthesized with an N-terminal spacer consisting of the sequence CWSG. PEG (20 kDa methoxy poly(ethylene glycol) maleimido-proprionamide (Chirotech Technology, Ltd., Cambridge, UK) was dissolved in a 25% acetonitiie:75% water solution. LPA peptide was added to a 5% molar excess over the PEG. The reaction was allowed to react overnight at RT. The maleimide group on the PEG couples covalently to the Cys residue on the peptide. Excess peptide was removed by size exclusion chromatography on a G25 column eluted with water. The conjugate was lyophilized.

Cell Culture:

MCF-7 and HT-29 cells were cultured in DMEM-F12 medium containing 5% fetal bovine serum (FBS), 5 μg/ml insulin, 1% amphopthericin B, 100 μg/ml streptomycin and 100 U/ml penicillin until they were 80% confluent. The cells were washed twice with 100 mM phosphate saline buffer (PBS), pH 7.2 and cultured for additional 2 days in the same medium but without FBS (basal medium). Cells were washed as described before and cultured in basal medium containing the leptin peptides of the present invention or controls as described above.

### Example 2

### Leptin Induced Increase in Leukemia Inhibitory Factor and its Receptor by Human Endometrium is Partially Mediated by Interleukin 1 Receptor Signaling

### Results:

More often than not many differences that may be observed in response to cytokine treatments are related to different cell types. To provide a broader perspective primary human endometrial epithelial and stromal cells (EEC and ESC) and epithelial cell lines (HES and Ishikawa) can be utilized to demonstrate treatment effects.

Leptin-Induced Effects on p-STAT3, LIF-R and LIF Levels in Human Endometrial Cell Cultures.

To analyze leptin-induced effects on p-STAT3, LIF-R and LIF levels in human endometrial cell cultures, after confirming via Western that the primary EEC and ESC and endometrial cell lines utilized herein have OB-R, leptin-induced effects on the levels of phosphorylated STAT3 can be evaluated. A leptin-induced increase in the levels of p-STAT3 is expected to be observed in EEC. However, the increase is expected to be dose-dependent. Similar effects are expected to be observed in the ESC and the Ishikawa and HES cell lines.

An increase in the levels of LIF-R after treatment of primary endometrial cells and endometrial cell lines with leptin can be determined by Western blot analysis. An increase in LIF-R levels (e.g., dose dependent) is indicative of a leptin induced response in EEC, HES and Ishikawa cells.

Likewise, treatment with leptin is expected to result in an increased level of LIF in primary EEC and is indicative of a leptin induced response. Similar patterns are expected after leptin treatment of ESC, HES and Ishikawa cells. An increase in LIF levels (e.g., dose dependent) is indicative of a leptin induced response in EEC, HES and Ishikawa cells.

### Inhibition of Leptin-Induced Effects by OB-R Antagonists

Western blot analysis is expected to reveal that treatment of Ishikawa cells with leptin will increase the levels of p-STAT3 over the non-treated controls. The leptin antagonists of the present invention, SEQ ID NOs: 34 and 35, (at about 30—about 300 nM) are expected to inhibit the leptin-induced increase, whereas scramble sequences are not expected to have an effect. Interestingly, leptin antagonist treatment may result in a decrease level of p-STAT3 when compared to the levels observed in the non-treated control cells. In a similar pattern, the levels of LIF-R are expected to be elevated over control following treatment with leptin. Co-treatment with the leptin antagonists of the present invention (at about 0.3—about 300 nM) are expected to reduce the leptin-induced increase which is indicative of inhibition of a leptin induced response. As before, treatment with scramble sequences are expected to have no effect on LIF-R levels when added in combination with leptin.

Treatment with leptin has been shown to increase the levels of LIF in the conditioned medium of cultured Ishikawa cells. The levels of LIF found in the conditioned medium following treatment with leptin in the presence of the scrambled peptide or IgG are not expected to be different than those from treatment with leptin alone. However, treatment of Ishikawa cells with both leptin and inhibitors of OB-R (either antibody or the leptin antagonists of the present invention) are expected to result in reduced LIF levels which would be indicative of inhibition of a leptin induced response. Similar results are expected to be found in primary EEC. The inhibition of leptin-induced effects on LIF levels by OB-R antibody or the leptin antagonists of the present invention in ESC may or may not be as dramatic as that observed in the Ishikawa cells.

Treatment of EEC with leptin has been shown to result in elevated levels of IL-1β and IL-1Ra. The leptin induced increase in IL-1β and IL-1Ra levels is expected to be abrogated by co-treatment with the leptin antagonists of the present invention and not affected by scrambled sequences. Leptin is expected to also increase the levels of IL-1R tl and, again, the leptin antagonists of the present invention are expected to inhibit the leptin-induced effects. An analogous effect is expected to be observed in ESC and Ishikawa cells.

### The Blockade of IL-1R tl Inhibits Leptin-Induced Effects on LIF-R Levels.

Treatment with IL-1β has been shown to increase the levels of LIF-R found in Ishikawa cells in a dose dependent manner. Moreover, when Ishikawa cells are treated with a combination of IL-1β and leptin (e.g., about 10 nM) a further increase of LIF-R levels has been observed in comparison to the cells treated with IL-1β alone. The leptin-induced increase of LIF-R levels in Ishikawa cells is expected to be abrogated by antagonists/inhibitors of IL-1R tl. A leptin-induced increase in LIF-R levels is expected to be partially blocked by the addition of IL-1Ra or anti-IL-1R tl antibody. No effect is likely to be found when the antibody is substituted by non-specific species-matched IgGs.

### Materials and Methods:

### Reagents and Antibodies

Goat polyclonal anti-human LIF-Rq anti OB-R (anti-NH₂ terminal end of human OB-R) and monoclonal anti-IL-1R type I (IL-1R tl) antibodies, human recombinant leptin, IL-1β and IL-1Ra can be obtained from R&D Systems Inc., MN. Antibodies for STAT3 (F-2) and phosphorylated STAT3 (p-STAT3, B-7) and non-specific mouse and goat IgGs can be obtained from Santa Cruz Biotechnology, Inc., Santa Cruz, Calif. Anti-vimentin, anti-cytokeratin and anti-CD45 antibodies can be obtained from Dako Corporation, Carpinteria, Calif. Human recombinant insulin can be obtained from Sigma Chemical Co., St Louis, Mo. Fetal bovine serum (FBS) can be obtained from Gemini Bioproducts, Woodland, Calif., and Dulbecco's modified Eagle's medium (DMEM/F-12) and antibiotic-antimycotic mixture can be obtained from GIBCO BRL Products, Gaithersburg, Md. Other chemicals can be obtained from Sigma Chemical Co., St Louis, Mo.

### Endometrial Tissues

Endometrial tissues can be obtained, for example, from hysterectomies of non-malignant etiologies under an IRB at, for example, the Vincent Center for Reproductive Biology (BRR— Massachusetts General Hospital, Boston, Mass.). Tissues can be digested with proteases for endometrial cell isolation as described elsewhere (Gonzalez et al., Hum. Reprod. 14: 2485-2492 (1999)). Briefly, endometrial biopsies can be minced and treated with collagenase I (0.1%)—DNAse I (0.005%) for 1 h at 37° C. After gland sedimentation endometrial stromal cells (ESC) can be separated from supernatants. EEC can be purified of ESC and macrophage contaminants by repeated incubation at 37° C. in a Falcon flask. Stromal and epithelial cell dispersions can be counted in a haemocytometer and cell viability (≧90%) can be assessed by optical microscopy using the Trypan Blue exclusion method. To assess homogeneity of cell preparations several specific monoclonal antibodies can be used in cell smears (Gonzalez et al., Hum. Reprod. 14: 2485-2492 (1999)), i.e., anti-vimentin (ESC+), anti-cytokeratin (EEC+) and anti-CD45 (leukocyte+). Homogeneity of cell preparations are preferred to be higher than 98%.

### Cell Cultures and Treatments.

Primary human endometrial cells (ESC and EEC) can be cultured for 5-9 days in DMEM-F12 medium containing 5% fetal bovine serum (FBS), 5 μg/ml insulin, 1% amphopthericin B, 100 μg/ml streptomycin and 100 U/ml penicillin until they are 80% confluent. Two types of endometrial epithelial cell lines, HES (originally derived from a benign proliferative endometrium) and Ishikawa (human endometrial epithelial adenocarcinoma, ECACC 9832301, Wiltshire, England) can be cultured under the same conditions described above. The cells can be washed twice with 100 mM phosphate saline buffer (PBS), pH 7.2 and cultured for additional 2 days in the same medium but without FBS (basal medium). Cells can be then washed as described before and cultured in basal medium containing leptin (e.g., 0, 3, 10 or 62 nM) or IL-1β (e.g., 0.6, 3 and 10 nM).

The anti-OB-R antibody (e.g., 10 and 20 μg/ml), a specific inhibitor of OB-R (e.g., 3, 120, 300 nM or 30 μM), and the leptin antagonists of the present invention can be used to assess whether leptin-induced effects are regulated primarily by the OB-R. Non-specific species-matched IgGs to the OB-R antibody and a scrambled version of the leptin antagonists of the present invention can serve as negative controls. Inhibitors of IL-1R can be used (monoclonal antibody anti-IL-1R tl and IL-1Ra) to assess whether leptin activated IL-1 induced signaling pathways or just activated similar components responsible for LIF/LIF-R expression. Treatment with the cytokine, inhibitor or the two combined can be implemented for, e.g., 5, 15, 30 and 60 min, and 24 and 48 h. The cells are prepared for Western blot analysis. The conditioned media is collected, lyophilized and stored at −80° C. until analysis for LIF, IL-1β and IL-1Ra by ELISA can be performed. Duplicate wells can be run for each treatment and the experiments repeated at least three times with different cell preparations.

### Cell Lysates

Endometrial cells can be washed with ice-cold PBS and lysed by, e.g., homogenization on ice with lysis buffer A [20 nM Tris, pH 7.4, containing 137 nM NaCl, 2 mM EDTA, 10% glycerol, 50 mM β-glycerophosphate, 1% Nonidet P-40 and a mixture of proteases and phosphatase inhibitors composed of 100 μM antipain, 0.1 mg/ml trypsin inhibitor, protease inhibitor cocktail 1:50 (Sigma), 50 nM NaF, 2 mM phenyl-methylsulfonyl fluoride, and 2 mM sodium orthovanadate]. Cellular lysates are centrifuged, for example, at 2400 g at 4° C. for 10 min. For nuclear lysates the cells can be scraped from the culture plates and homogenized with, e.g., lysis buffer B (10 nM Tris, pH 7.4, 0.25 M sucrose, 0.1 mM EDTA containing the same mixture of protease and phosphatase inhibitors as buffer A). Protein concentrations can be determined using the Bradford protein assay (BioRad Laboratories Inc., Hercules, Calif.).

### Immunoprecipitation

Protein from nuclear or cellular lysates can be incubated in ependorf tubes containing primary antibodies (anti-LIF-R, OB-R, STAT3 and p-STAT3 diluted in buffer A) for 2 h at 4° C. under constant stirring. The immuno-complexes can be incubated with of Protein G-Agarose (Amersham Pharmacia Biotech) diluted 1:1 with buffer A for a 2 additional hours under constant stirring. The beads can then be centrifuged and washed with buffer A containing, e.g., 0.5 M NaCl followed by a final wash with buffer A.

### Western Blot

Cellular lysates and their immunoprecipitates containing, e.g., 10-30 μg of protein plus Laemmli buffer (1:1) can be incubated at about 95° C. for about 5 min. Electrophoresis can be performed at 220 V for 5 min followed by 130 V for 45 min (BioRad, electrophoresis apparatus) on 7.5% (for STAT3, p-STAT3, LIF-R and IL-1R tl) and 10% (for OB-R) SDS-PAGE gels. Nuclear lysates can also be used to detect p-STAT3. Electroblotting onto 0.2 μm nitrocellulose membranes can be performed at, e.g., 22 V overnight at 4° C. in 48 nM Tris-39 nM glycine buffer containing 0.037% SDS and 20% methanol. Membranes can be washed with, e.g., 20 mM Tris, 137 mM NaCl pH 7.4 buffer containing 0.5% Tween 20 (v/v) (wash buffer) and incubated for 1 h at room temperature in blocking buffer containing low fat dried milk (5%, w/v) in wash buffer. The membranes can subsequently be incubated at room temperature for 1 h with 2 μg/ml of anti OB-R, LIF-R and 1 μg/ml of IL-1R tl, STAT3 and p-STAT3 antibodies in blocking buffer. After washing, the membranes can be incubated for, e.g., 30 min in wash buffer containing about 2.5% normal horse or rabbit serum (Vector Laboratories). Immune-complexes can be detected with biotinylated horse anti-mouse or rabbit anti-goat antibodies (Vector Laboratories) followed by incubation with streptavidin-horseradish peroxidase-conjugate (Amersham) for about 30 min at room temperature. Specific bands in the blots can be visualized using an ECL-chemiluminescent assay (Amersham) and Imagetek-B film (American X-ray & Medical Supply, Rancho Cordoba, Calif.). Non-specific mouse and goat IgGs (Santa Cruz Biotechnology), for example, can be used instead of primary antibodies to produce negative control blots.

To quantitatively assess the effects of cytokines and inhibitors of OB-R and IL-1R tl on the antigen expression, the blots can be scanned and analyzed (with, e.g., TotalLab version 2003.02, NonLinear Dynamics Ltd, or equivalent).

### Determination of LIF, IL-1β and IL-1Ra Secretion by Human Endometrial Cells

Conditioned media from cells cultured under the experimental conditions described above can be used to quantify the secretion of LIF, IL-1β and IL-1Ra by ELISA (Quantikine®, R&D Systems). The experiments can be replicated one, two, three or more times and standards, controls, and samples can be assayed in duplicate. Cytokine concentrations as determined by ELISA are expected to be within the range of the standard curve and expressed in, e.g., pg/ml/mg protein. The intra- and interassay coefficients of variation are expected to be between 5-8% and 8.5-10%, respectively. The lowest detectable concentration of each cytokine by ELISA is expected to be: LIF (about 9 pg/ml), IL-1 (about 0.11 pg/ml) and IL-1Ra (about 15.6 pg/ml), similar to those reported by the manufacturer. LIF-ELISA is reported by the manufacturer to have a sensitivity of less than 8 pg/ml for both natural and recombinant human LIF and 100% specificity (no significant cross-reactivity or interference with a diversity of human and mouse cytokines and growth factors is expected to be observed). The performance characteristics for human IL-1β and IL-1Ra ELISAs reported are expected to be: 100% specificity, sensitivity 0.1 pg/ml and 14 pg/ml, respectively, based on prior work by the Applicants.

### Statistical Analysis

A one-way ANOVA test with Dunnett error protection and a confidence interval of 95% from the Analyse-it for Microsoft Excel (Leeds, UK, htpp://www.analyse-it.com), or similar, can be used for data analysis. Data expressed as mean±SEM. Values for p<0.05 are to be considered statistically significant.

### Example 3

### A Peptide Derived from the Human Leptin Molecule is a Potent Inhibitor of the Leptin Receptor Function in Rabbit Endometrial Cells

### Expression of Functional Ob-R by Rabbit Endometrial Cells and Binding of the Leptin Antagonists of the Present Invention to Ob-R.

After the purification steps, homogenous preparations of rabbit endometrial stromal (rESC) and epithelial cells (rEEC) can be successfully obtained. Immunocytochemical studies are expected to show that rESC expressed vimentin but not cytokeratin. In contrast, rEEC has not been shown to express vimentin but has been shown to be positive for the epithelial cell marker, cytokeratin.

Interestingly, both cellular types have been shown to constitutively express OB-R. Addition of leptin or anti-OB-R antibody to cultures of rESC or rEEC have been shown to not affect the expression of OB-R. The leptin antagonists of the present invention are also not expected to affect the expression of OB-R. Negative controls using cells incubated with non-specific species-matched IgGs are expected to show no staining for any of the antigens tested.

Western blot analyses of lysates from cell cultures are expected to corroborate the immunocytochemical findings. A main band of approximately 190 kDa corresponding to the full length of OB-R is expected to be found in all protein lysates from rESC and rEEC cultured in basal medium or medium containing the tested compounds. Competitive binding studies between human leptin and the leptin antagonists of the present invention that show that ¹²⁵I-leptin bound to OB-R from cellular lysates is displaced by addition of the leptin antagonists of the present is indicative of the ability of the leptin antagonists of the present invention to inhibit leptin induced responses.

Up-Regulation of p-STAT3 Expression by Leptin in Rabbit Endometrial Cells and Inhibition by Anti-Ob-R Antibody and the Leptin Antagonists of the Present Invention.

To test the functionality of OB-R expressed by the rabbit endometrial cells, p-Stat3 phosphorylation can be investigated following leptin treatment. The basal expression of p-Stat3 is expected to be up-regulated by leptin. Similar results are expected to be obtained for rEEC.

Inhibition of the leptin up-regulation of Stat3 phosphorylation by the leptin antagonists of the present invention is indicative of the ability of the leptin antagonists of the present invention to inhibit leptin up-regulation of Stat3. For the leptin antagonists of the present invention blockade of p-Stat3 expression is expected to appear to be specific and likely to be found at very low concentrations of the leptin antagonists of the present invention and is not expected to be observed with scrambled sequences at the same concentrations. Negative controls using cells incubated with non-specific species matched IgGs are expected to show no staining for any of the antigens tested.

Results from western blot determinations of p-Stat3 expression by rESC and rEEC are expected to confirm the immunocytochemical findings. An 81 kDa band corresponding to p-Stat3 known to be found in samples from rESC and rEEC. Both cell types are known to exhibit increased p-Stat3 expression after incubation with leptin.

The antibody specific for the NH₂ terminal end of human OB-R has been shown to effectively block the receptor function in the cell cultures by decreasing the leptin up-regulation of Stat3 phosphorylation in rESC and rEEC. This effect has been shown to be specific since the cells incubated with non-specific goat IgGs are not expected to down-regulate p-Stat3 after leptin treatment.

Inhibition of basal levels of expression of p-Stat3 in rESC and rEEC is indicative of the ability of the peptides of the present invention to function as antagonists of this physiological process. No inhibitory effect on p-Stat 3 expression is expected to be seen when the cells are incubated with scrambled sequences.

### Leptin Regulation of IL-1R tl Expression by Endometrial Rabbit Cells.

Results from immunocytochemical and western blot studies on leptin regulation of IL-1R tl expression and the antibody and the leptin antagonists of the present invention are expected to show effects that are similar in rESC and rEEC. Rabbit endometrial cells cultured in basal medium are known to weakly express IL-1R tl. Leptin has been shown to substantially up-regulate IL-1R tl expression. Moreover, as is found for p-Stat3 expression, the leptin effect on IL-R tl expression is expected to be reduced of abolished by the addition of the leptin antagonists of the present invention. Cells incubated with non-specific mouse IgGs are not expected to show any positive staining for IL-1R tl.

A western blot analysis of IL-1R tl expression from the cellular lysates of rESC cultures has been shown to confirm the immunocytochemical results. The incubation of rESC cultures with anti-OB-R antibody can be used as a positive control for the OB-R inhibition. The increase in intensity of the 80 kDa band corresponding to IL-1R tl in the blot from protein extracts of rESC following incubation of the cells with leptin (e.g., 3 nM) compared to cells cultured in basal medium has been shown to demonstrate the leptin up-regulation of IL-1R tl expression. The addition of anti-OB-R antibody has been shown to neutralize the leptin up-regulation of IL-1R tl, further demonstrating the specificity of the leptin effect. Furthermore, incubation of rESC with non-specific goat IgGs and leptin has been shown to not prevent the leptin up-regulation of IL-1R tl expression.

The leptin antagonists of the present invention are expected to exhibit inhibitory effects on leptin up-regulation of IL-R tl expression in rESC cultures similar to those found with anti-OB-R antibody. The leptin antagonists of the present invention are expected to reduce or inhibit the basal expression of IL-1R tl by rESC. Moreover, dose inhibition (e.g., dose dependent) of leptin up-regulation of IL-1R tl by the leptin antagonists of the present invention is indicative of the inhibitory and specificity of the leptin antagonists of the present invention.

In contrast, scrambled sequences are not expected to alter the leptin effect on IL-1R tl expression. Results from western blot of rEEC lysates are expected to be similar.

### Leptin Regulation of LIF Secretion and LIF-R Expression by Rabbit Endometrial Cells.

The effects of leptin on LIF-R expression in rESC and rEEC can also be investigated. It has been shown that leptin up-regulates the expression of LIF-R in both cell types. The basal expression of LIF-R is known to be increased by leptin. Once again, the incubation of rESC cultures with leptin plus the leptin antagonists of the present invention are expected to show that these peptides also reduce or inhibit the leptin effect on LIF-R expression. Negative controls for LIF-R staining with rESC incubated with non-specific goat IgGs are expected to exhibit no staining.

Western blot analysis of rESC and rEEC lysates for LIF-R can also be performed. Leptin at 3 nM has been shown to significantly increase the expression of LIF-R relative to the basal level. The addition of anti OB-R antibody to rEEC cultures containing leptin completely has been shown to inhibit LIF-R expression in contrast to the negative control, consisting of cells incubated with non-specific goat IgGs. The leptin antagonists of the present invention are expected to reduce or totally inhibit the leptin effect on LIF-R expression at, for example, 30 and 120 nM doses. Incubation of rEEC with scrambled sequences is not expected to prevent the leptin up-regulation of LIF-R.

LIF secretion by rabbit endometrial cell cultures is known to be low but detectable by the ELISA kit. LIF concentrations can be divided by the milligrams of total protein present in the cell lysates and expressed as pg/ml/mg protein.

Secretion of LIF by rEEC has been shown to be higher than in the case of rESC under all-experimental conditions. Moreover, leptin has been shown to increase LIF secretion in a dose-dependent manner. Moreover, the blockade of OB-R with the OB-R antibody has been shown to abolish the leptin effect.

The addition of the leptin antagonists of the present invention at doses from, for example, 0.3 to 300 nM is expected to significantly inhibit the leptin (e.g., 3 nM) induction of LIF secretion by rEEC. A higher leptin dose (e.g., 10 nM) is expected to result in a further increase in LIF secretion by rEEC which is expected to be effectively inhibited by the addition of equimolar concentrations of the leptin antagonists of the present invention.

### Materials and Methods

### Hormones, Antibodies, and Reagents.

Human recombinant leptin, monoclonal antibodies anti IL-1R tl, anti LIF-R, and goat polyclonal antibody to the NH2 terminal end of human OB-R can be provided by R & D System Inc., MN. Monoclonal antibody (B-7) for phosphorylated Stat3 (p-Stat3) and non-specific mouse and goat sera can be obtained from Santa Cruz Biotechnology, Inc., Santa Cruz, Calif. Anti-vimentin, anti-cytokeratin (pan-cytokeratin) and anti-CD45 antibodies are from Dako Corporation, Carpinteria, Calif. ¹²⁵I-human leptin can be obtained from New England Nuclear (NEN, MA), and human recombinant insulin and MCDB-105 culture medium can be obtained from Sigma Chemical Co., St Louis, Mo. Fetal bovine serum (FBS) can be obtained from Gemini Bioproducts, Woodland, Calif., and Dulbecco's modified Eagle's medium (DMEM) and antibiotic-antimycotic mixture can be provided by GIBCO BRL Products, Gaithersburg, Md. Other chemicals are obtainable from Sigma.

### Design of the Leptin Antagonists of the Present Invention.

The design of the leptin peptide antagonists (LPA) of the present invention is described above in Example 1.

### Synthesis and Purification of LPAs

The synthesis and purification of the leptin antagonists of the present invention are described in Example 1, above. In brief, they were synthesized by solid-phase peptide synthesis (Applied Biosystems, Model 431A Peptide Synthesizer) using 9-fluorenylmethoxycarbonyl (fmoc) chemistry. The peptides were purified using a C18 preparative high performance liquid chromatography (HPLC) column.

Because the leptin antagonist peptides of the present invention proved to be sparingly soluble in water, dimethyl-sulphoxide (DMSO) was used as solvent for the preparation of concentrated solutions of these peptides. After solubilization with DMSO it is then possible to dilute the peptides either in 50 mM Tris and 100 mM NaCl, pH 8.5 to promote proper re-folding or directly in culture medium to obtain the desired concentrations for the binding and cell culture experiments.

### Protein Analytical Procedures

The purities of the leptin antagonists of the present invention and scrambled sequences were evaluated by reversed phase HPLC. Circular dichroism spectroscopy was used to assess secondary structures. The molecular masses of the peptides were determined by mass spectral analysis on a Voyager-RP Biospectrometer MALDI-TOF Workstation (Perseptive Biosystems, Cambridge, Mass.). Spectra were averages of approximately 200 scans.

### Endometrial Tissues

Uteri from non-mated female New Zealand white rabbits can be obtained from usual sources. The rabbit uteri used for preparation of endometrial cells should be obtained under an approved IACUC protocol. Endometrial tissues can be scraped from the uteri and then digested with proteases for isolation of endometrial cells as described elsewhere (Simon et al., Endocrinology 134: 521-528 (1994)). Briefly, endometrial tissues can be minced and treated with collagenase I (0.1%)—DNAse I, 0.005% for 1 h at 37° C. rEEC can be purified of rESC and macrophage contaminants by repeated incubation for 1-2 h at 37° C. in a Falcon flask. rEEC and rESC dispersions can be counted in a haemocytometer and cell viability can be assessed by optical microscopy using the Trypan Blue exclusion method. The mean of cell viability should be, preferably, higher than 95%. The homogeneity of cell preparations can be assessed by the expression of cytokeratin (EEC+), vimentin (ESC+) and CD45 (leukocytes+) using specific antibodies (Simon et al., Endocrinology 134: 521-528 (1994)). Homogeneity of cell preparations should be, preferably, approximately 98%.

### Cell Cultures

Rabbit endometrial cells (5×10⁵ cells/well) can be cultured for 5-9 days in, for example, DMEM-MCDB105 (3:1) medium containing 10% fetal bovine serum (FBS), 5 μg/ml insulin, 1% amphopthericin B, 100 μg/ml streptomycin and 100 U/ml penicillin until confluent layers are obtained. The cells can then be washed twice with 100 mM phosphate saline buffer (PBS)—2% BSA (w/v), pH 7.2 and cultured for an additional 2 days in the same medium but without FBS (basal medium). This procedure is performed to reduce any effects of cytokines from FBS on the phosphorylation rate of Stat3 as well as the expression of other cytokines and their receptors. Cells can then be washed as described before and cultured in basal medium containing, for example, leptin (0-10 nM), OB-R antibody (1-20 μg/ml) and/or leptin antagonists of the present invention (0-300 nM). Cultures can then be stopped at 24 h and the cells can be used for immunocytochemistry, binding studies and western blot analysis. The conditioned media can then be collected, lyophilized and stored at −80° C. for ELISA determinations. Duplicate wells can be run for each treatment and the experiments repeated at least three times with different cell preparations from different rabbits. Controls should be the same cellular preparations cultured in basal medium containing non-specific goat IgGs and/or scrambled leptin sequences.

### Determination of LIF Secretion by Rabbit Endometrial Cell Cultures

IL-1β from rabbits and humans are known to have sequence differences, but no data is currently available concerning the sequence of rabbit LIF. In addition, there are no commercial kits for measuring rabbit IL-1β or LIF. However, because LIF has been suggested to be important for mammalian reproduction, to measure LIF in rabbit culture supernatants using a quantitative method designed for determining human LIF has been attempted. See, U.S. Pat. Nos. 7,409,929 and 7,612,043, which are incorporated herein by reference. In brief, these patents report conditioned media (n=3 per treatment) from rESC and rEEC cultures can be used to quantify the secretion of LIF by ELISA (LIF-Quantikine®, R&D Systems). Standards, controls and samples can be assayed in duplicate. The intra- and interassay coefficients of variations have been shown to be from about 0.7-12% and from about 3-7%, respectively (U.S. Pat. Nos. 7,407,929 and 7,612,043). According to the manufacturer, the performance characteristics of the ELISA are as follows: 100% specificity and sensitivity less than 8 pg/ml for both natural and recombinant human LIF; no significant cross-reactivity or interference is observed with a great diversity of human and mouse cytokines and growth factors.

### Immunocytochemistry:

The rabbit endometrial cells can be cultured, for example, as described above in duplicate on 8-well glass-bottom culture plates (Nalgene Nunc International, Naperville, Ill.) and fixed, for example, with methanol at −20° C. for 20 min for immunocytochemical studies. All the antibodies tested can then be diluted in PBS-2% BSA (w/v; buffer A).

The expression of OB-R can be assessed by incubation of rEC for 1 h at room temperature with goat antibodies directed towards the amino terminal ends of human OB-R(R&D system) diluted 1:80 in buffer A.

Anti-IL-1R tl (1 μg/ml), anti-LIF-R (2 μg/ml), anti-vimentin (1:50), anti-cytokeratin (1:50) and anti-p-Stat3 (1 μg/ml) antibodies can be used to assess the expression of the respective antigens by rEEC and rESC cultures. After incubation with primary antibodies the cells can then be incubated with a streptavidin-biotin-alkaline phosphatase system according to the manufacturer directions (Vectastain, ABC-AP kit, Vector Laboratories, Burlingame, Calif.) and counter-stained with hematoxylin (Dako). Negative controls can include cell preparations in which the primary antibodies are substituted by irrelevant species matched IgGs.

### Preparation of Cell Lysates.

After culture in the presence of leptin, LPAs or antibodies, the endometrial cells are then washed with ice-cold PBS and lysed by homogenization on ice with lysis buffer [e.g., 20 nM Tris, pH 7.4, containing 137 nM NaCl, 2 mM EDTA, 10% glycerol, 50 mM β-glycerophosphate, 50 nM NaF, 1% Nonidet P-40, 2 mM phenyl-methylsulfonyl fluoride, 2 mM sodium orthovanadate, 100 μM antipain, 0.1 mg/ml trypsin inhibitor and protease cocktail inhibitor 1:50 (Sigma)]. Cellular lysates are then centrifuged at 24,000 g at 4° C. for 10 min. Protein concentrations are then determined using the Bradford protein assay (BioRad Laboratories Inc., Hercules, Calif.).

### Western Blot Analysis

Protein extracts from cell lysates can be combined (1:1) with Laemmli buffer (2× concentrated) and 10 μg of proteins are loaded per lane on 7.5% (for p-Stat3, IL-1R tl and LIF-R) and 10% (for OB-R) SDS-PAGE gels. Electrophoresis can be performed at 65 V for 1-1.5 h (BioRad, electrophoresis apparatus). Electroblotting onto 0.2 μm nitrocellulose membranes can be performed at 22V overnight at 4° C. in 48 nM Tris-39 nM glycine buffer containing 0.037% SDS and 20% methanol. Membranes can be blocked, for example, for 1 h at room temperature in 20 mM Tris, 137 mM NaCl pH 7.4 buffer containing 0.5% Tween 20 (v/v) (wash buffer) supplemented with 5% Amersham blocking reagent (blocking buffer) and rinsed three times with wash buffer. The membranes can subsequently be incubated at room temperature for 1 h with 2 μg/ml of anti OB-R, IL-1R tl, LIF-R and p-Stat3 antibodies in blocking buffer. Detection can be performed by incubation with biotinylated anti-mouse or anti-goat antibodies followed by incubation with streptavidin-horseradish peroxidase-conjugate (Amersham Pharmacia Biotech) for 30 min at room temperature. Positive specific antigen-antibody reactions in the blots are visualized using an ECL-chemiluminescent assay (Amersham) followed by exposure on KODAX X-Omat AR film (IBI-Kodak Ltd, Cambridge, U.K.). Non-specific mouse and goat IgGs (Santa Cruz Biotechnology) can be used instead of primary antibodies to produce negative control blots.

### Binding Assays

¹²⁵I-human leptin can serve as ligand and LPA-2 and LPA-2Sc as competitors for the binding to OB-R in cellular extracts. All compounds are then diluted in assay buffer (50 mM Tris and 100 mM NaCl, pH 8.5) to promote proper re-folding. Each assay tube can contain, for example, 50 μl of cellular lysate (100 μg protein from 10⁶ cells cultured for two days in basal medium), 50 μl of ¹²⁵I-human leptin (100,000 cpm), and 50 μl of LPAs (providing 10⁻⁵-10⁻¹⁴ M/tube). The tubes can then be incubated for 24 h at room temperature. Then, 250 μl of 1% BSA (w/v) and 500 μl of 20% polyethylene glycol (w/v) in PBS can be added and the tubes incubated for an additional 12-h period at 4° C. The tubes can then be centrifuged at 12,000 g for 15 min at 4° C. After careful aspiration of supernatant and complete drying of the tube-walls with filter paper, ¹²⁵I-human leptin binding in the precipitates can be measured (Beckman 5000, counter). The binding of ¹²⁵I-leptin to OB-R in absence of LPAs is then set at 100%, and LPA competitive binding values are then expressed as percentages of total cpm. Affinity binding constants for LPAs can then be calculated from the competition curves.

### Statistical Analysis

A one-way ANOVA test with Dunnett error protection and a confidence interval of 95% is typically used for data analysis (e.g., Analyse-it for Microsoft Excel (Leeds, UK, htpp://www.analyse-it.com). Data expressed as, e.g., mean±SEM. Values for P<0.05 will be considered statistically significant.

### Example 4

### Mouse Embryo Implantation Requires Endometrial Leptin Signaling

### Impact of Ob-R Inhibitors on Mouse Embryo Implantation.

It has been determined in previous work (U.S. Pat. Nos. 7,407,929 and 7,612,043) that the vehicle solution did not have any toxic effect on implantation in the treated mice. Analysis of implantation sites in mouse endometria after mating has shown that a single injection of the leptin antagonists of the present invention (e.g., 3 μM/10 μl) at, for example, Day 3 of pregnancy to significantly impair implantation as compared to negative controls (scrambled sequences).

It has been shown (U.S. Pat. Nos. 7,407,929 and 7,612,043) that intra-uterine injection of anti-OB-R specific antibodies (1.5 μg/horn) showed no reduction of implantation rate. However, higher doses of the blocking OB-R antibody (7.5 μg/horn) negatively affected mouse implantation in contrast to the control horns treated with non-specific goat IgGs.

The embryos that implant in horns treated with the leptin antagonists of the present invention (prophetically) or anti-OB-R antibodies are expected to be mainly found in the distal portion of the uterine horn—furthest from the OB-R inhibitor injection site. In contrast, the implanted embryos in the scrambled sequences, vehicle or IgG treated mice are expected to be found uniformly distributed throughout the uterine horn.

### The Effects of Ob-R Inhibitors on the Development of Mouse Embryos In Vivo

Analysis of recovered embryos after intra-uterine injections of OB-R inhibitors is expected to show that the development of unimplanted embryos is significantly reduced or arrested in horns treated with the leptin antagonists of the present invention or anti OB-R antibodies.

### Expression of Ob-R, LIF-R, IL-1R tl and β3-Integrin by Mouse Endometrium

OB-R is known to be mainly found in endometrial epithelial cells (lumen and glands) from non-pregnant mice. After implantation occurred (Day 6 and 10 of pregnancy) OB-R staining is only detected in some epithelial glands. Treatment with the leptin antagonists of the present invention is not expected to affect the expression of OB-R in vivo by mouse endometrium.

Treatment with the leptin antagonists of the present invention is expected to negatively decrease the levels of LIF-R and IL-1R tl. By immunohistochemical analysis the β3-integrin has been shown to be found mainly in EEC (lumen and glands) in pregnant (Day 6 and 10 of pregnancy) and non-pregnant mouse endometrium. Intra-uterine injection of the leptin antagonists of the present invention is expected to decrease the expression of β3-integrin by mouse endometrium. No effect on β3-integrin staining is expected to be found in the uterine horns treated with scrambled sequences or controls. Similar results on decrease of β3-integrin staining are expected to be found in control mouse uterine horns treated with OB-R antibodies.

The leptin antagonists of the present invention are expected to significantly decrease the expression of IL-1R tl, LIFR and β3 integrin at the expected time of embryo implantation in the mouse endometrium.

To substantiate an association of IL-1R or LIFR and integrin β3 in vivo, double-fluorescent confocal laser scanning microscopy on mouse endometrium can be conducted. The cellular distribution and the relative intensity of fluorescence on the cells can be assessed qualitatively. Immunofluorescence of Alexa fluor 593 for IL-1R and of Alexa Fluor 488 localizing anti-integrin β3 mAb is expected to be reveal a strong membrane-associated granular pattern for each of the molecules. Using double-fluorescent labeling, colocalization of IL-1R tl and integrin β3 in the same cells is expected to be indicated by yellow fluorescence. Similar results are expected to be found for the colocalization of LIFR and B3 integrin. The colocalization of both cytokine receptors by lacking proper combination of primary and secondary antibodies need not be determined. The same problem is encountered in the double-fluorescent staining of either IL-1R, LIFR and OBR. This last receptor can be detected with goat anti-mouse antibodies.

### Materials and Methods

### Chemicals

Armenian hamster antibody anti-β3 integrin (N-20, mouse origin), mouse anti-Armenian hamster IgGs, rabbit polyclonal antibodies again the carboxy terminus of LIF-R (C-19, human or mouse origin), IL-1 receptor type I (IL-1R tl, antibody M-20) and their respective blocking peptides for competition studies can be obtained from Santa Cruz Biotechnology, Inc., Santa Cruz, Calif. Goat polyclonal anti-mouse OB-R (AF497, anti-NH2 terminal end of mouse OB-R), non-specific rabbit and goat IgGs can be obtained from R&D Systems Inc., MN. Other chemicals can be obtained from Sigma Chemical Co., St. Louis, Mo. Normal goat and rabbit sera and biotinylated horse anti-mouse/anti-rabbit and rabbit anti-goat IgGs antibodies can be obtained from Vector Laboratories (Vector Laboratories, Burlingame, Calif.). Biotinylated goat anti rabbit IgGs antibodies (ALI3409, mouse IgG adsorbed) can be obtained from BioSource International, Camarillo, Calif. Alexa Fluor 594 goat anti-rabbit IgG, Alexa Fluor 488 goat-anti mouse IgG and DAPI (4′,6-diamino-2 phenylindole, dihydrochloride) can be obtained from Molecular Probes Inc., Eugene, Oreg.

### OB-R Inhibitors

The leptin antagonists of the present invention and scrambled versions of these peptides (negative control) are synthesized as described above in Example 1. Stock solutions are in DMSO. Stock solutions are then diluted with phosphate-buffered saline (PBS) and sterile-filtered to produce the desired leptin antagonist working solutions for the in vivo experiments. The final DMSO concentration in the vehicle is preferably about 0.04%.

Goat anti-mouse OB-R antibodies and normal goat IgGs (negative control) from R & D Systems Inc. diluted in PBS can be sterile filtered and then used to assess the impact of blockade of OB-R signaling on mouse implantation.

### Animals

Virgin, 8-10 wk-old, female, C57BL6 mice (Charles River Laboratories, Wilmington, Mass.) can be housed in suitable animal facilities in accordance with NIH standards for the care and use of experimental animals. The room should be provided with a controlled temperature range (22-24° C.) on a 14L:10D cycle. Mice should be given water and food ad libitum.

Superovulation can be induced in female mice (n=40) by an i.p. injection of 10 IU pregnant mare serum gonadotrophin (PMSG) (Sigma Chemical Company, St Louis, Mo.) followed by 10 IU hCG (Sigma) 48 h later. Next, the female mice are then mated with fertile males of the same strain to induce pregnancy. The following morning, the females exhibiting vaginal copulatory plugs should be separated for the next experiments. The day of vaginal plug was recorded as Day 1 of pregnancy. Mice should be weighed before treatment and before they are euthanized.

### Surgical Procedures and Intra-Uterine Blocking Treatments

Each mouse can be labeled with an earring tag to record treatment and any observation pre or post treatment. Forty-eight hours after hCG administration (Day 3 of pregnancy), female mice with documentation of vaginal plug are then anaesthetized by i.p. injection of Avertin (2,2,2 tribromoethanol, 30 μl/g body weigh; Sigma). A surgical incision is then made on the dorsal mid-line through the skin, and each uterine horn with fat pad and ovary is then pulled-out of the body cavity using forceps (Roboz, Gainthersburg, Md.). Under a dissecting microscope (Cambridge Laboratories) different compounds are then injected in each uterine horn. Each compound is then delivered slowly using a Hamilton syringe (Hamilton Co., model PC010) holding an in-house made glass needle (<27G; capillary pipette 20 μl, Unopette, Becton Dickinson). The glass needle allows the observation that the compounds are then effectively injected into each horn. The surgical incision is then closed with metal auto clips (9 mm, Becton Dickinson). Mice are then placed in warmed cage (slide warmer, Fisher) and returned to the animal facilities after full recovery from the anesthetic.

On Day 3 of pregnancy, about 10 to about 15 μl of the leptin antagonists of the present invention or a scrambled sequence (3.3 μM) can be delivered into the lumen of the proximal region of the right horn of each pregnant mouse (n=15). Each animal can serve as its own internal control, with the left uterine horn receiving the vehicle. For positive control, a group of mice can receive 15 μl goat anti OB-R antibodies at doses of 1.5 and 7.5 μg in the right horn and non-specific goat IgG solutions in the left horn. Concentrations of OB-R inhibitors used in vivo are then calculated from results of previous experiments in endometrial cell cultures [Gonzalez and Leavis, Endocrine 21: 185-195 (2003)]. The effects of surgical treatment, intra-uterine injection and toxicity of vehicle solutions on implantation are then assessed in about 8 pregnant mice that received intra-uterine injections of PBS and PBS-DMSO.

To analyze the effects of OB-R inhibitors on pregnancy and expression of several molecules by the endometrium the mice will be euthanized by cervical dislocation on Day 6 and 10 of pregnancy.

On Day 6 of pregnancy the mice receive an intra-ocular injection of 0.1 ml of 1% Evans Blue dye in saline 5 min before euthanasia on the morning of day 6 after mating. Uteri from these mice are then examined for implantation sites by observing a localized increase of uterine vascular permeability (Paria et al., 2001). The uteri are then extracted and the implantation sites are then counted in each horn. The numbers of implanted embryos in the right (experimental) and left (control) uterine horns are then counted under a dissecting microscope. Uteri without blue bands from 6-day pregnant mice are then flushed to recover unimplanted blastocysts. The uteri from 10-day pregnant mice are then examined with the naked eye for implantation masses. The histology of uteri with decidual capsules (implantation site) are then examined for evidence of embryological development. A portion of each uterine horn is then dissected and treated to produce cryostat sections or fixed in 4% paraformaldehyde in PBS and embedded to produce paraffin blocks.

### Immunohistochemical Determinations

To assess the potential effects of blockade of endometrial OB-R function in vivo on the expression of various cytokine receptors, immunohistochemistry in cryostat and paraffin block sections (4 μm) can be performed. Unmasking of several antigen epitopes (β3-integrin, LIF-R and IL-1R tl) in paraffin sections can be performed. Briefly, samples can be boiled for 10 min in a pressure cooker containing 10 mM sodium citrate/1 mM ethylene-diamino tetracetic acid (EDTA), pH 6 solution. After quenching endogenous peroxidase activity with H₂O₂ (3% water solution) and blocking (2.5% horse or rabbit normal serum), tissue sections are then incubated for 1 h at room temperature with the following primary antibodies diluted in PBS-0.1% BSA: anti-β3 integrin, IL-1R tl, LIF-R and OB-R antibodies (all at 4 μg/ml). Biotinylated secondary antibodies anti-primary antibody species are then used. The tissues are then incubated with a streptavidin-biotin-peroxidase system according to the manufacturer's directions (Vectastain, ABC-AP kit, Vector Laboratories, Burlingame, Calif.), counter-stained with hematoxylin (Dako Corporation, Carpinteria, Calif.) and mounting with VectaMount (Vector Labs) for permanently preserve staining from the precipitable enzyme substrate. Negative controls include tissue preparations in which the primary antibodies are substituted by irrelevant species matched IgGs. Negative controls for competitive studies with anti IL-1R tl and LIF-R antibodies are then generated by pre-incubation with their respective blocking peptides (20 μg/ml, Santa Cruz Biotechnology). All washing steps can be performed by immersion of the preparations 3 times in PBS for 5 min at room temperature.

Results from immunohistochemistry preferably analyzed blind by a single examiner. In addition, a limited number of immunohistochemical preparations can be examined twice by the same observer or by an additional observer. The number of endometrial stromal and epithelial cells (glands or lumen) positively stained (from a total number of 100 cells) for each antigen and preparation is to be recorded in representative fields selected in a random manner. Staining intensity is then assigned using a semiquantitative HSCORE as is described by Lessey et al (Lessey et al., J. Clin. Invest. 90: 188-195 (1992)). The HSCORE is then calculated using the following equation: HSCORE=ΣPi(i+1), where “i” is the intensity of staining within a value of 1, 2 or 3 (weak, moderate and strong, respectively) and “Pi” is the percentage of stained cells for each intensity varying from 0 to 100% (Lessey et al., J. Clin. Invest. 90: 188-195 (1992)).

### Confocal Laser Scanning Microscopy

Confocal laser scanning microscopy can be performed in serial sections from paraffin blocks to determine the colocalization of receptors (OB-R, IL-1R tl and LIF-R) and β3 integrin within endometrium tissue from non-pregnant and pregnant mice after intra-uterine treatments. The protocols for immunostaining to be used are similar to those already described for regular color-based immunohistochemistry but the secondary antibodies and counterstaining agent to be used are changed. Alexa Fluor 594 goat anti-rabbit IgG (red fluorescence) and Alexa Fluor 488 goat anti-mouse IgG (green fluorescence) conjugates can be used to detect LIF-R and IL-1R tl, and β3 integrin, respectively. In addition, a second FITC-conjugated antibody (rabbit anti-goat IgG-FITC) can be used to detect OB-R. The double-fluorescent stained specimens are then analyzed with a confocal laser scanning microscope equipped with an external argon laser (BioRad). To avoid photobleaching of fluorochromes during fluorescence microscopy, the slides can be embedded in anti-fade solution (Dako). For nuclear and chromosome counterstaining a 300 nM DAPI PBS-dimethylformamide (DMF) solution can be used.

We claim:

1. A composition comprising an amino acid sequence consisting essentially of NH₂-IDNIRTVITKILTKXDDQV-CONH₂ [SEQ ID NO.: 34], wherein X is selected from a group consisting of T, I, L and V.

2. The composition of claims 1, wherein the amino acid sequence is composed of D-amino acids.

3. The composition of claims 1, wherein the amino acid sequence is composed of L-amino acids.

4. The composition of claims 1, wherein said amino acid sequence is attached to a PEG (polyethylene glycol) moiety.

5. The composition of claim 4, wherein PEG moiety is attached to said amino acid sequence by an amino acid spacer.

6. The composition of claim 5, wherein said amino acid spacer consists of the amino acid sequence GSWC [SEQ ID NO.: 33].

7. comprising administering to a mammal a therapeutically effective amount of the composition of claim 1.

8. The method of claims 7, wherein the reduction or inhibition of ObR signaling in the cell results in a reduction or inhibition of the up-regulatory effects of leptin on a signaling event downstream of ObR.

9. The method of claim 8, wherein the signaling event downstream of the leptin receptor is selected from a group consisting of p-Stat-3, β3-integrin, IL-1 and LIF signaling.

10. The method of claim 7, wherein the reduction or inhibition of ObR signaling results in a reduction or inhibition of the up-regulatory effects of leptin on the expression of a leptin-sensitive target.

11. The method of claim 10, wherein the leptin-sensitive target is selected from the group consisting of p-Stat-3, β3-integrin, IL-1R tl, LIF-R, LIF, IL-1β, and IL-1Ra.

12. The method of claims 7, wherein the mammal is selected from the group consisting of humans, mice, and rabbits.

13. The method of claims 7, wherein the administration of the composition results in the reduction of the likelihood of embryo implantation.

14. A composition comprising an amino acid sequence consisting essentially of NH₂-VHLLDRLNELXNSIQIVN-CONH₂ [SEQ ID NO.: 35], wherein X is selected from a group consisting of D, I, L and V.

15. The composition of claims 14, wherein the amino acid sequence is composed of D-amino acids.

16. The composition of claims 14, wherein the amino acid sequence is composed of L-amino acids.

17. The composition of claims 14, wherein said amino acid sequence is attached to a PEG (polyethylene glycol) moiety.

18. The composition of claim 17, wherein PEG moiety is attached to said amino acid sequence by an amino acid spacer.

19. The composition of claim 18, wherein said amino acid spacer consists of the amino acid sequence GSWC [SEQ ID NO.: 33].

20. A method for reducing or inhibiting ObR signaling in a leptin-responsive cell, the method comprising administering to a mammal a therapeutically effective amount of the composition of claim 14.

21. The method of claims 20, wherein the reduction or inhibition of ObR signaling in the cell results in a reduction or inhibition of the up-regulatory effects of leptin on a signaling event downstream of ObR.

22. The method of claim 21, wherein the signaling event downstream of the leptin receptor is selected from a group consisting of p-Stat-3, β3-integrin, IL-1 and LIF signaling.

23. The method of claim 20, wherein the reduction or inhibition of ObR signaling results in a reduction or inhibition of the up-regulatory effects of leptin on the expression of a leptin-sensitive target.

24. The method of claim 23, wherein the leptin-sensitive target is selected from the group consisting of p-Stat-3, β3-integrin, IL-1R tl, LIF-R, LIF, IL-1β, and IL-1Ra.

25. The method of claims 20, wherein the mammal is selected from the group consisting of humans, mice, and rabbits.

26. The method of claims 20, wherein the administration of the composition results in the reduction of the likelihood of embryo implantation


SEQUENCE LISTING (35 sequences)

SEQ ID NO: 1 - protein, 32 aa, Homo sapiens
IQKVQDDTKTLIKTIVTRINDISHTQSVSSKQ

SEQ ID NO: 2 - protein, 26 aa, Homo sapiens
SRNVIQISNDLENLRDLLHVLAFSKS

SEQ ID NO: 3 - protein, 26 aa, Artificial Sequence
  Description of Artificial Sequence Synthetic peptide
VAEVLNRSDLIQRISFSLDLNNSKLH

SEQ ID NO: 4 - protein, 31 aa, Homo sapiens
QKVQDDTKTLIKTIVTRINDISHTQSVSSKQ

SEQ ID NO: 5 - protein, 29 aa, Homo sapiens
VQDDTKTLIKTIVTRINDISHTQSVSSKQ

SEQ ID NO: 6 - protein, 27 aa, Homo sapiens
DDTKTLIKTIVTRINDISHTQSVSSKQ

SEQ ID NO: 7 - protein, 27 aa, Homo sapiens
VQDDTKTLIKTIVTRINDISHTQSVSS

SEQ ID NO: 8 - protein, 23 aa, Homo sapiens
VQDDTKTLIKTIVTRINDISHTQ

SEQ ID NO: 9 - protein, 21 aa, Homo sapiens
VQDDTKTLIKTIVTRINDISH

SEQ ID NO: 10 - protein, 19 aa, Homo sapiens
VQDDTKTLIKTIVTRINDI

SEQ ID NO: 11 - protein, 17 aa, Homo sapiens
VQDDTKTLIKTIVTRIN

SEQ ID NO: 12 - protein, 22 aa, Homo sapiens
IQISNDLENLRDLLHVLAFSKS

SEQ ID NO: 13 - protein, 20 aa, Homo sapiens
ISNDLENLRDLLHVLAFSKS

SEQ ID NO: 14 - protein, 18 aa, Homo sapiens
NDLENLRDLLHVLAFSKS

SEQ ID NO: 15 - protein, 16 aa, Homo sapiens
LENLRDLLHVLAFSKS

SEQ ID NO: 16 - protein, 22 aa, Homo sapiens
SRNVIQISNDLENLRDLLHVLA

SEQ ID NO: 17 - protein, 20 aa, Homo sapiens
SRNVIQISNDLENLRDLLHV

SEQ ID NO: 18 - protein, 18 aa, Homo sapiens
NVIQISNDLENLRDLLHV

SEQ ID NO: 19 - protein, 16 aa, Homo sapiens
IQISNDLENLRDLLHV

SEQ ID NO: 20 - protein, 10 aa, Homo sapiens
DLENLRDLLH

SEQ ID NO: 21 - protein, 19 aa, Artificial Sequence
  Description of Artificial Sequence Synthetic peptide
VQDDTKTLIKTIVTRINDI

SEQ ID NO: 22 - protein, 19 aa, Artificial Sequence
  Description of Artificial Sequence Synthetic peptide
VQDDAKTLIKTIVTRINDI

SEQ ID NO: 23 - protein, 19 aa, Artificial Sequence
  Description of Artificial Sequence Synthetic peptide
VQDDIKTLIKTIVTRINDI

SEQ ID NO: 24 - protein, 19 aa, Artificial Sequence
  Description of Artificial Sequence Synthetic peptide
VQDDDKTLIKTIVTRINDI

SEQ ID NO: 25 - protein, 19 aa, Artificial Sequence
  Description of Artificial Sequence Synthetic peptide
VITQDTINVKRTDKITLDI

SEQ ID NO: 26 - protein, 18 aa, Artificial Sequence
  Description of Artificial Sequence Synthetic peptide
NVIQISNDLENLRDLLHV

SEQ ID NO: 27 - protein, 18 aa, Artificial Sequence
  Description of Artificial Sequence Synthetic peptide
NVIQISNALENLRDLLHV

SEQ ID NO: 28 - protein, 18 aa, Artificial Sequence
  Description of Artificial Sequence Synthetic peptide
NVIQISNNLENLRDLLHV

SEQ ID NO: 29 - protein, 18 aa, Artificial Sequence
  Description of Artificial Sequence Synthetic peptide
NVIQISNILENLRDLLHV

SEQ ID NO: 30 - protein, 18 aa, Artificial Sequence
  Description of Artificial Sequence Synthetic peptide
QINLSLLDHVNIRDNVEL

SEQ ID NO: 31 - protein, 19 aa, Artificial Sequence
  Description of Artificial Sequence Synthetic peptide
IDNIRTVITKILTKTDDQV

SEQ ID NO: 32 - protein, 18 aa, Artificial Sequence
  Description of Artificial Sequence Synthetic peptide
VHLLDRLNELDNSIQIVN

SEQ ID NO: 33 - protein, 4 aa, Artificial Sequence
  Description of Artificial Sequence Synthetic peptide
GSWC

SEQ ID NO: 34 - protein, 19 aa, Artificial Sequence
  Description of Artificial Sequence Synthetic peptide
IDNIRTVITKILTKXDDQV

SEQ ID NO: 35 - protein, 18 aa, Artificial Sequence
  Description of Artificial Sequence Synthetic peptide
VHLLDRLNELXNSIQIVN